(19) United States
(12) Patent Application Publication
(10) Pub. No.: US 2018/0121625 A1
(43) Pub. Date: May 3, 2018

(54) METHOD AND SYSTEM FOR FACILITATING WOUND CARE MANAGEMENT

(71) Applicants: BRET ALAN BARKER, (US); UBERWOUNDCARE, INC, San Jose, CA (US)

(72) Inventor: BRET ALAN BARKER, SALINAS, CA (US)

(21) Appl. No.: 15/803,305

(22) Filed: Nov. 3, 2017

Related U.S. Application Data
(60) Provisional application No. 62/417,196, filed on Nov. 3, 2016.

(51) Int. Cl. G06F 19/00; A61B 5/00

(52) U.S. Cl. CPC G06F 19/3418; G06F 19/321; A61B 5/445; G06F 19/322; G06F 19/327

(57) ABSTRACT

Disclosed is a method of facilitating wound care management of a subject. The method includes receiving a request for wound management from a user device. Further, the method includes receiving one or more images of a wound of the subject from the user device. Yet further, the method includes analyzing the one or more images of the wound. Moreover, the method includes determining one or more medical conditions of the wound based on the analyzing. Further, the method includes generating one or more work schedules for one or more healthcare-providers based on at least one of the request and the one or more medical conditions. Yet further, the method includes transmitting the one or more work schedules to one or more healthcare-provider devices associated with the one or more healthcare-providers.

### FIELD OF THE INVENTION

The present invention relates to telemedicine. In particular, the present invention relates to a method and a system of allowing communication between medical providers and patients through video and cloud based services.

### BACKGROUND OF THE INVENTION

A root cause analysis of why there are over 6.5 million wounds yearly in America and is growing has identified a need to provide more frequent hands-on wound care, provider to patient communication, and ability to readily assess wounds and modify treatments or provide referral ability quickly.

Oftentimes, a patient may not make an appointment due to transportation issues, pain, dread or other psychological factors. Decisions made by patients regarding self-wellness may be misplaced. The patient may believe “it's okay” or “it's not that bad,” and delay treatment as a result of difficulty, challenges, or inconvenience of wound treatment as viewed by the patient, client or customer.

Certain healthcare processes of reporting systems allow the visiting staff to self-report their visit attendance, which at times causes miscommunication, fraud, and abuse by the visiting licensed crowd sourced providers, intentionally or unintentionally. The administrative staff of a typical care agency is faced with monitoring the off-site personnel by spot-checking visit attendance data or relying on patient complaints or feedback. With on demand on-location visits, tracked by global positioning (GPS) on mobile devices, tied to time and date stamped visit check in by provider, images taken during the wound assessment and management episode, along with real-time charting in the electronic medical record (EMR), by the on demand on-location provider, these disadvantages are minimized and readily identified with reporting functions within the various modules of the cloud based wound management system.

Another disadvantage to such self-reporting procedures is that the reporting is generally self-documented by visiting staff on paper reports. A full time visiting staff employee can perform over 1,250 visits a year, which could require a typical administrative staff person to spend an average of five minutes or more per employee visit to process and enter the information into appropriate coding/billing, scheduling, and payroll systems. This can be inefficient and costly.

Accordingly, there is a need for a method and system that provides for improved monitoring, reporting, data communication, and/or tracking of information relating to field service personnel such as visiting staff in the home healthcare field.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

Disclosed is a method of facilitating wound care management of a subject. The method includes receiving, using a communication device, a request for wound management from a user device. Further, the method includes receiving, using the communication device, one or more images of a wound of the subject from the user device. Yet further, the method includes analyzing, using a processing device, the one or more images of the wound. Moreover, the method includes determining, using the processing device, one or more medical conditions of the wound based on the analyzing. Further, the method includes generating, using the processing device, one or more work schedules for one or more healthcare-providers based on at least one of the request and the one or more medical conditions. Yet further, the method includes transmitting, using the communication device, the one or more work schedules to one or more healthcare-provider devices associated with the one or more healthcare-providers.

According to some embodiments, a system for facilitating wound care management of a subject is disclosed. The system includes a communication device and a processing device. The communication device is configured for receiving a request for wound management from a user device. Further, the communication device is configured for receiving one or more images of a wound of the subject from the user device. Yet further, the communication device is configured for transmitting one or more work schedules to one or more healthcare-provider devices associated with one or more healthcare-providers. Moreover, the processing device is configured for analyzing the one or more images of the wound and determining one or more medical conditions of the wound based on the analyzing. Further, the processing device is configured for generating the one or more work schedules for the one or more healthcare-providers based on at least one of the request and the one or more medical conditions.

According to some embodiments, a mobile application telemedicine based on-demand on-location crowd sourced licensed provider wound care management system is disclosed. The mobile application and cloud based server in communication with cloud based mobile application on provider and patient mobile devices which adheres to patient privacy laws through video and data encryption. The mobile application and cloud based server includes mobile applications for provider scheduling, location tracking and travel management, on-location visit recording and plan of care plan administration, and privacy compliant video and text chat communications.

Further, the application and related components of the system are cloud-based to allow patients to communicate directly with providers on mobile devices using the application for focused on-location wound care services using [crowd-sourced] or local agency staff.

According to some embodiments, a method is disclosed for formulating and rendering wound management care plans, collecting and reporting information from field based personnel, data collection from client/customer via the client/customer mobile device (mobile to mobile), and other functions pertaining to the client/customer visit, including the clients use of mobile applications and self-reporting of wound data and follow up care with the mobile application, self-care, educational materials, ability to utilize the system.

The present disclosure also provides for scheduling, location tracking, and delivery of medical supplies to the treatment site. The objective of the present invention is to allow patients to communicate directly with providers on mobile devices using the application, for focused, on-location, wound care services.

The on-demand, on-location healthcare business is relatively new in the over $30B wound care sector of the healthcare industry. Instead of requiring patients to undergo prolonged hospital stays or frequent visits to a clinic, a telemedicine based on demand, on-location agency brings the medical services of wound assessment via telemedicine, digital photography and computerized analysis of wound size and composition, along with management of the wound to the patient's location. Payment for services rendered is primarily paid by federal and state Medicare and Medicaid programs, private insurance carriers, and private pay. Patient well-being often depends on the visit and attendance compliance of the visiting nurse, trips to the primary care provider, wound clinic or other wound services which may be delayed and/or rescheduled, leading to worsening of a chronic wound, thereby delaying and preventing wound closure, otherwise termed healing.

According to some aspects, an easily and readily accessible network of providers, physicians, physician assistants, nurse practitioners, nurses, physical therapists is provided. Various care givers may assess and manage wounds remotely via telemedicine, support hands-on wound management and assessment on-location on demand, as the patient/client/customer may request according to preference. With many cries of a nursing shortage for years, there are over 3.44 million licensed nurses in the United States, 810,000 licensed physicians; about 200,000 of the licensed nurses have some type of wound care certification. In contrast, there are roughly 1,000 standalone wound clinics in the United States, and about 800 wound specific departments within facilities such as hospitals and has yet to reduce the number of chronic wounds yearly. Meeting the patient where they are, providing ready and private communication, shipping supplies directly to their location and providing wound assessment, management and training on wound self-care, is an ideal method to directly target the problem where it lies.

Further, utilizing privacy law compliant telemedicine and mobile application, various healthcare agencies, such as home health agencies, dispatch nurses or physical therapists to the homes, and physician assistant or nurse practitioner if needed, of patients to perform required healthcare assessments, tasks, and other vital services for wound management. The frequency and length of time of a visit and the care provided by the visiting professional are important to obtaining a positive outcome and improving the health of the patient with focused wound assessments completed on-location initially by patient mobile device to telemedicine provider and followed through by on site provider and frequent contact with patient via telemedicine application, reduced onsite time and lower overall costs can be achieved while increasing patient contact, reducing waste, reducing healing time and lessening the chance of infection related hospital admissions. Contact coordinated between home health, discharge planners, long term care, primary care providers and others provides readily accessible data to providers of the patient within privacy laws. An example of cost reduction is when Government reimbursement to a home healthcare agency is paid on a per episode of sickness basis; therefore, the visiting nurse is often required to recommend the frequency and type of visits by a licensed crowd sourced providers. Thus, it is important to ensure compliance by the licensed crowd sourced providers in attending the needed visits, and knowing what tasks and services are required for the specific patient. Tracking the duration of the actual visit is also important and is thus automated. Care agency administrators are then responsible for processing patient visit data records generated by the visiting staff to be transferred into coding/billing, scheduling, and payroll systems. Utilizing a fee per visit and episode type, incentives for on time and early wound healing reduce wastage, and provides patient focused care.

With GPS-reported locations and short wound care episode stays, cost containment, reduction in wastage, the disclosed system can facilitate focused patient care. This reduces the overall burden on the patient holistically, and facilitates overall cost reduction for third-party payers as well.

Further, the present disclosure describes embodiments related to systems and methods of data collection, reporting and tracking of in-field home healthcare personnel. However, it is understood that the present invention may encompass and apply to various systems and methods and is intended to relate to alternative embodiments for use in communicating information with, and the monitoring of, any type of field service personnel.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of a platform consistent with various embodiments of the present disclosure.

FIG. 2 is a block diagram of a system for facilitating wound care management of a subject, in accordance with some embodiments.

FIG. 3 illustrates a flowchart of a method of facilitating wound care management of a subject, in accordance with some embodiments.

FIG. 4 illustrates a flowchart of a method of communicating with multiple health care providers, in accordance with some embodiments.

FIG. 5 illustrates a flowchart of a method for confirming one or more work schedules, in accordance with some embodiments.

FIG. 6 illustrates a flowchart of a method for processing an electronic form, in accordance with some embodiments.

FIG. 7 illustrates a flowchart of a method for generating routes for the one or more healthcare-providers, in accordance with some embodiments.

FIG. 8 illustrates a flowchart of a method for analyzing a wound, in accordance with some embodiments.

FIG. 9 illustrates a flowchart of a method for obtaining treatment instructions for a wound, in accordance with some embodiments.

FIG. 10 is a simplified block diagram of a wound management system illustrating representative modules, according to some embodiments.

FIG. 11 is a block diagram of a wound management system illustrating communication among various modules, according to some embodiments.

FIG. 12 is a block diagram of the wound management system illustrating communication among various modules, according to some embodiments.

FIG. 13 is a flow chart of a method 1300 for initiating and establishing bi-directional communication with a communication device, a remote mobile application and a cloud based server, in accordance with some embodiments.

FIG. 14 is a flow chart of an office administrative method as part of the on location telemedicine supported wound management system, in accordance with some embodiments.

FIG. 15 is a flow chart of a field personnel visitation method associated with a communication device and related tracking as part of the on location telemedicine supported wound management system, in accordance with some embodiments.

FIG. 16A shows a user interface displaying an icon corresponding to the mobile application, in accordance with some embodiments.

FIG. 16B shows a user interface during an initialization procedure, with the splash screen displayed, in accordance with some embodiments.

FIG. 16C shows a user interface after initialization procedure completes, in accordance with some embodiments.

FIG. 17A shows a user interface corresponding to set pickup location feature, in accordance with some embodiments.

FIG. 17B shows a user interface containing a registration form, in accordance with some embodiments.

FIG. 17C shows a user interface with a form that allows a user to enter credit card data, in accordance with some embodiments.

FIG. 17D shows a user interface depicting a pickup location and a drop-off location, in accordance with some embodiments.

FIG. 18A shows a user interface depicting the menu of nurses and physicians, in accordance with some embodiments.

FIG. 18B shows a user interface depicting an example of nurses, showing credentials, photo and distance, in accordance with some embodiments.

FIG. 18C shows a user interface depicting an example of provider physician, showing credentials, photo and distance, in accordance with some embodiments.

FIG. 19A shows a user interface depicting a portal logon screen to electronic medical record, in accordance with some embodiments.

FIG. 19B shows a user interface depicting a portrait view of a medical record, in accordance with some embodiments.

FIG. 19C shows a user interface depicting a landscape view of the mobile access to the medical record, in accordance with some embodiments.

FIG. 20A shows a user interface depicting a selection screen, in accordance with some embodiments.

FIG. 20B shows a user interface depicting types of services, in accordance with some embodiments.

FIG. 20C shows a user interface depicting details about an order wound consultation, in accordance with some embodiments.

FIG. 20D shows a user interface depicting an order for a webinar on wound care, in accordance with some embodiments.

FIG. 21A shows a user interface depicting a registration form for a waiting room, in accordance with some embodiments.

FIG. 21B shows a user interface depicting a logon screen, in accordance with some embodiments.

FIG. 21C shows a user interface depicting a patient waiting for a provider, in accordance with some embodiments.

FIG. 21D shows a user interface depicting the acceptance of the provider, in accordance with some embodiments.

FIG. 21E shows a user interface depicting a chat message feature, in accordance with some embodiments.

FIG. 22A shows a user interface for a web application depicting a waiting room patient entry, in accordance with some embodiments.

FIG. 22B shows a user interface for a web application depicting a provider screen remote computer, in accordance with some embodiments.

FIG. 22C shows a user interface for a web application depicting a waiting room patient listing, in accordance with some embodiments.

FIG. 23A shows a user interface for a mobile application depicting a login screen, in accordance with some embodiments.

FIG. 23B shows a user interface depicting a photo of a wound on a particular day, in accordance with some embodiments.

FIG. 23C shows a user interface depicting a login screen for accessing web tissue analysis reports, in accordance with some embodiments.

FIG. 23D shows a user interface depicting a gallery of wound photos, in accordance with some embodiments.

FIG. 23E shows a user interface depicting analysis of a wound, in accordance with some embodiments.

FIG. 24 is a block diagram showing operating systems supported, in accordance with some embodiments.

FIG. 25 is a flowchart of a mobile device tracking method, in accordance with some embodiments.

FIG. 26 is a flowchart related to a visit recording method, in accordance with some embodiments.

FIG. 27 is a flowchart related to an image transmission method, in accordance with some embodiments.

FIG. 28 is a flowchart related to an image capture method, in accordance with some embodiments.

FIG. 29 illustrates an exemplary computing system that may be employed to implement processing functionality for various embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of provisioning telemedicine, embodiments of the present disclosure are not limited to use only in this context.

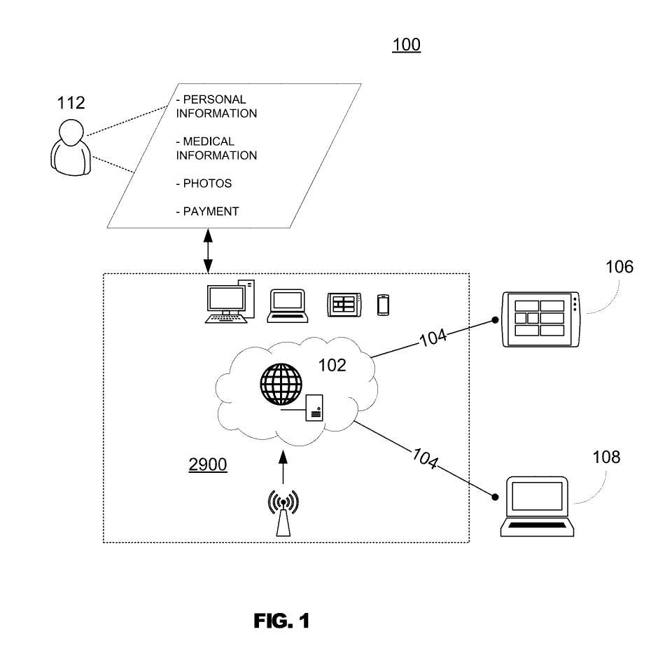

FIG. 1 is an illustration of a platform consistent with various embodiments of the present disclosure. By way of non-limiting example, the online platform 100 for provisioning telemedicine may be hosted on a centralized server 102, such as, for example, a cloud computing service. The centralized server 102 may communicate with other network entities, such as, for example, a mobile device 106 (such as a smartphone, a laptop, a tablet computer etc.) and other electronic devices 108 (such as desktop computers, etc.) over a communication network 104, such as, but not limited to, the Internet. Further, users of the platform may include one or more relevant parties such as, patients, care givers, nurses, therapists, doctors, insurance agents and administrators. Accordingly, electronic devices operated by the one or more relevant parties may be in communication with the platform.

A user 112, such as the one or more relevant parties, may access platform 100 through a software application. The software application may be embodied as, for example, but not be limited to, a website, a web application, a desktop application, and a mobile application compatible with a computing device 2900.

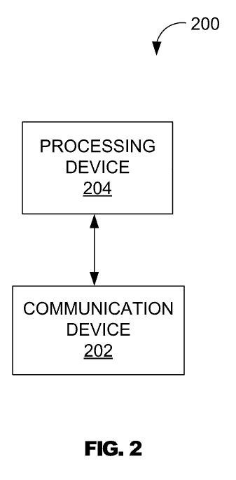

Turning to FIG. 2, a system 200 for facilitating wound care management of a subject, in accordance with some embodiments. As shown, the system 200 includes a communication device 202 and a processing device 204. The communication device 202 may be configured for receiving a request for wound management from a user device. Further, the communication device 202 may be configured for receiving one or more images of a wound of the subject from the user device. Yet further, the communication device 202 may be configured for transmitting one or more work schedules to one or more healthcare-provider devices associated with one or more healthcare-providers.

Further, the system 200 may include a processing device 204 configured for analyzing the one or more images of the wound. Yet further, the processing device 204 may be configured for determining one or more medical conditions of the wound based on the analyzing. Moreover, the processing device 204 may be configured for generating the one or more work schedules for the one or more healthcare-providers based on one or more of the request and the one or more medical conditions.

In some embodiments, the communication device 202 may be configured for transmitting a service request to multiple healthcare-provider devices associated with multiple health care providers. Further, the communication device 202 may be further configured for receiving one or more responses from one or more of the multiple healthcare-provider devices. The processing device 204 may be further configured for identifying the one or more healthcare-providers based on the one or more responses.

In some embodiments, the communication device 202 may be further configured for receiving tracking data from the one or more healthcare-provider devices associated with the one or more healthcare-providers. Further, the processing device 204 may be further configured for comparing one or more schedule parameters associated with the one or more work schedules with a corresponding tracking data and validating the one or more work schedules based on a result of the comparing.

In some embodiments, the communication device 202 may be further configured for transmitting an electronic form to the one or more healthcare-provider devices. The electronic form may be based on one or more of the request and the medical condition. The communication device 202 may be further configured for receiving form data from the one or more healthcare-provider devices. Further, the form data may be entered into the electronic form by the one or more healthcare-providers. Yet further, the processing device 204 may be further configured for analyzing the form data based on one or more rules. Further, validating the one or more work schedules may be further based on the analyzing of the form data.

In some embodiments, the communication device 202 may be further configured for receiving one or more current locations of the one or more healthcare-provider devices and transmitting one or more routes to the one or more healthcare-provider devices. Further, the processing device 204 may be further configured for generating one or more routes associated with the one or more work schedules based on the one or more current locations.

In some embodiments, the communication device 202 may be further configured for establishing an encrypted communication session between a healthcare-provider device and the user device based on one or more of the request and the medical condition.

In some embodiments, the processing device 204 may be further configured for determining one or more wound characteristics associated with the wound based on analysis of the one or more images of the wound. The one or more images may include representation of a reference object. Further, the analysis may be based on one or more physical characteristics of the reference object. The processing device 204 may be further configured for identifying one or more dressing products based on the one or more wound characteristics. Yet further, the processing device 204 may be configured for transmitting an indication of the one or more dressing products to one or more of the user device and the one or more healthcare-provider devices.

In some embodiments, the processing device 204 may be further configured for identifying treatment instructions based on one or more of the one or more wound characteristics and the medical condition. Further, the communication device 202 may be configured for transmitting the treatment instructions to one or more of the user device and the one or more healthcare-provider devices.

In some embodiments, the processing device 204 may be further configured for initiating dispatching of the one or more dressing products to a location associated with the subject.

In some embodiments, the processing device 204 may be further configured for determining a time schedule associated with the dispatching. Further, the time schedule may include indication of multiple times and multiple dressing products to be dispatched at the multiple times.

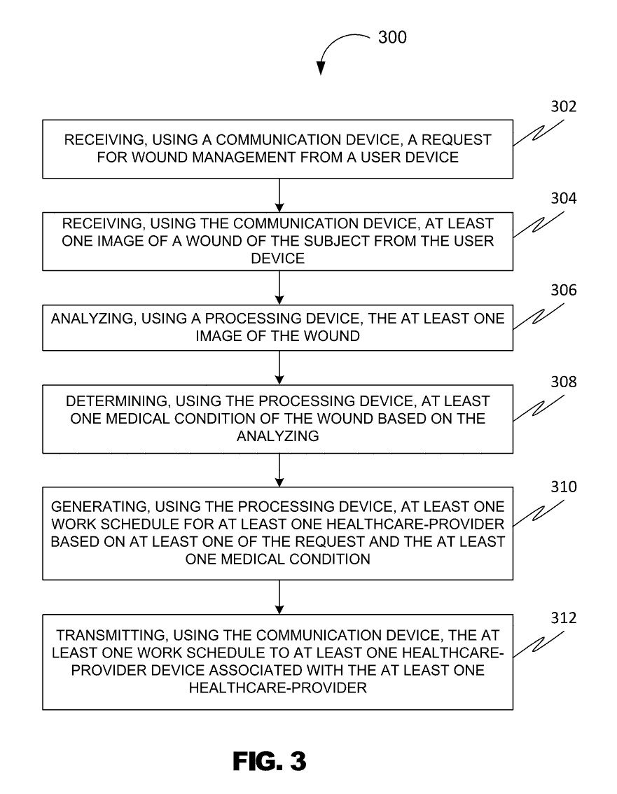

FIG. 3 illustrates a flowchart of a method 300 of facilitating wound care management of a subject, in accordance with some embodiments. At 302, the method 300 may include receiving, using a communication device (such as the communication device 202), a request for wound management from a user device. Further, at 304, the method 300 may include receiving, using the communication device, one or more images of a wound of the subject from the user device. Yet further, at 306, the method 300 may include analyzing, using a processing device (such as the processing device 204), the one or more images of the wound. Thereafter, at 308, the method 300 may include determining, using the processing device, one or more medical conditions of the wound based on the analyzing. Next, at 310, the method 300 may include generating, using the processing device, one or more work schedules for one or more healthcare-providers based on one or more of the request and the one or more medical conditions. Further, at 312, the method 300 may include transmitting, using the communication device, the one or more work schedules to one or more healthcare-provider devices associated with the one or more healthcare-providers.

In some embodiments, the method 400 may further include establishing, using the communication device, an encrypted communication session between a healthcare-provider device and the user device based on one or more of the request and the one or more medical conditions.

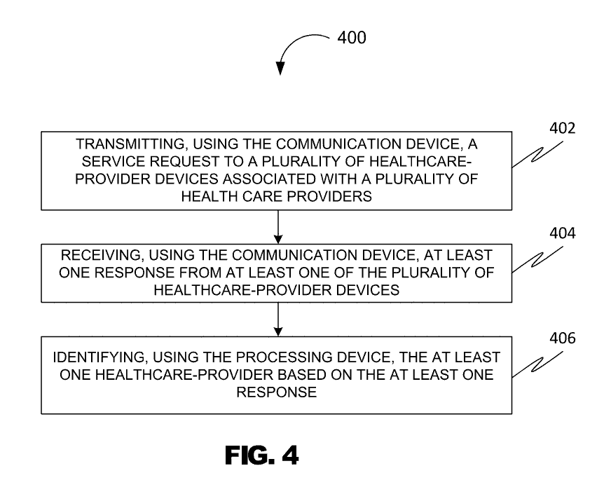

FIG. 4 illustrates a flowchart of a method 400 of communicating with multiple health care providers, in accordance with some embodiments. At 402, the method 400 may include transmitting, using the communication device, a service request to multiple healthcare-provider devices associated with the multiple health care providers. Further, at 404, the method 400 may include receiving, using the communication device, one or more responses from one or more of the multiple healthcare-provider devices. Thereafter, at 406, the method 400 may include identifying, using the processing device, the one or more healthcare-providers based on the one or more responses.

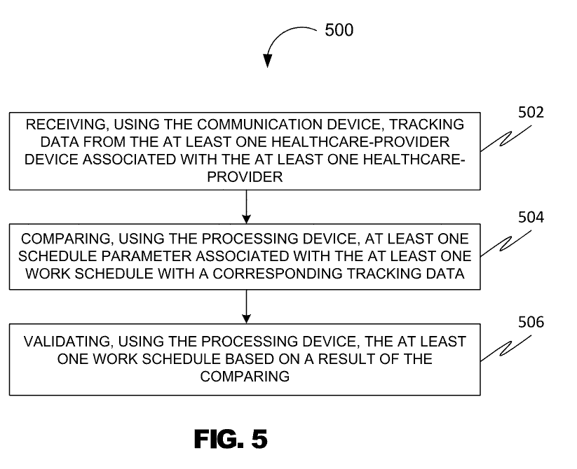

FIG. 5 illustrates a flowchart of a method 500 for confirming one or more work schedules, in accordance with some embodiments. At 502, the method 500 may include receiving, using the communication device, tracking data from the one or more healthcare-provider devices associated with the one or more healthcare-providers. Further, at 504, the method 500 may include comparing, using the processing device, one or more schedule parameters associated with the one or more work schedules with a corresponding tracking data. Finally, at 506, the method 500 may include validating, using the processing device, the one or more work schedules based on a result of the comparing.

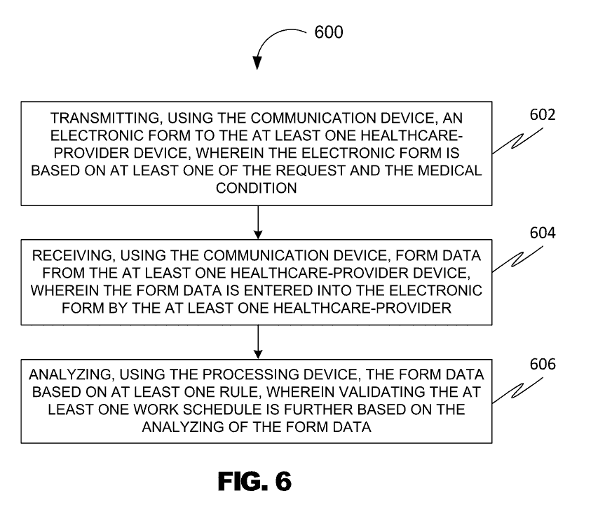

FIG. 6 illustrates a flowchart of a method 600 for processing an electronic form, in accordance with some embodiments. At 602, the method 600 may include transmitting, using the communication device, an electronic form to the one or more healthcare-provider devices. The electronic form may be based on one or more of the request and the medical condition. Further, at 604, the method 600 may include receiving, using the communication device, form data from the one or more healthcare-provider devices. The form data may be entered into the electronic form by the one or more healthcare-providers. Moreover, at 606, the method 600 may include analyzing, using the processing device, the form data based on one or more rules. The validating the one or more work schedules may be further based on the analyzing of the form data.

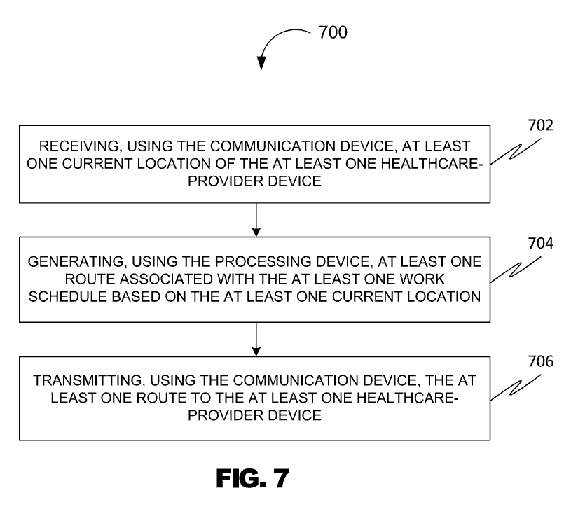

FIG. 7 illustrates a flowchart of a method 700 for generating routes for the one or more healthcare-providers, in accordance with some embodiments. At 702, the method 700 may include receiving, using the communication device, one or more current locations of the one or more healthcare-provider devices. Further, at 704, the method 700 may include generating, using the processing device, one or more routes associated with the one or more work schedules based on the one or more current locations. Yet further, at 706, the method 700 may include transmitting, using the communication device, the one or more routes to the one or more healthcare-provider devices.

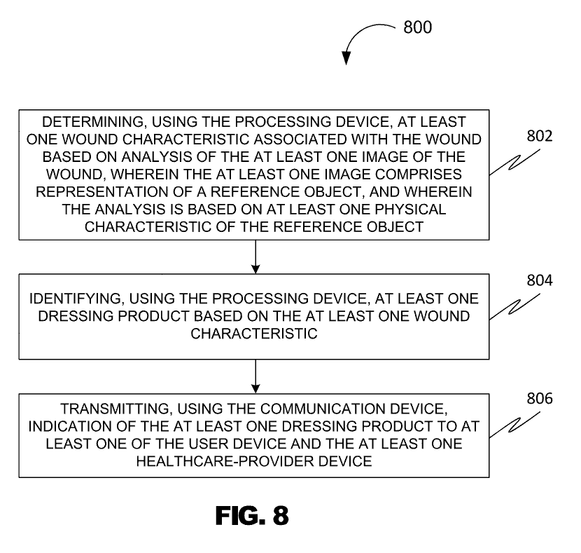

FIG. 8 illustrates a flowchart of a method 800 for analyzing a wound, in accordance with some embodiments. At 802, the method 800 may include determining, using the processing device, one or more wound characteristics associated with the wound based on analysis of the one or more images of the wound. Further, the one or more images may include representation of a reference object. The analysis may be based on one or more physical characteristic of the reference objects. Further, at 804, the method 800 may include identifying, using the processing device, one or more dressing products based on the one or more wound characteristics. Yet further, at 806, the method 800 may include transmitting, using the communication device, indication of the one or more dressing products to one or more of the user device and the one or more healthcare-provider devices. Further, the method 800 may include initiating, using the processing device, dispatching of the one or more dressing products to a location associated with the subject. In further embodiments, the method 800 may further include determining, using the processing device, a time schedule associated with the dispatching. Further, the time schedule may include indication of multiple times and multiple dressing products to be dispatched at the multiple times.

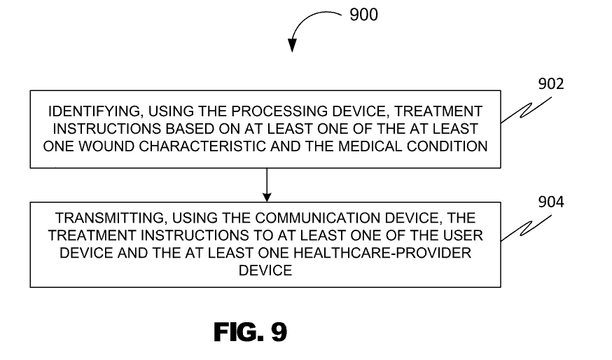

FIG. 9 illustrates a flowchart of a method 900 obtaining treatment instructions for a wound, in accordance with some embodiments. At 902, the method 900 may include identifying, using the processing device, treatment instructions based on one or more of the one or more wound characteristics and the medical condition. Thereafter, at 904, the method 900 may include transmitting, using the communication device, the treatment instructions to one or more of the user device and the one or more healthcare-provider devices.

According to some embodiments, the disclosed wound management system includes one or more modules including, but not limited to, a Base and Mobile Framework, a Scheduling Module, an Electronic Visit Record and Care Plan Module (EMR), a GPS Tracking and Travel Management Module, a Virtual Waiting Room and Video Messaging System, a Wound Care Management Module, a Supply Order Fulfillment Module, a Wound Analysis and Management Intelligence Module, an Administrative Center Module, and an Enterprise Management and Fulfillment System.

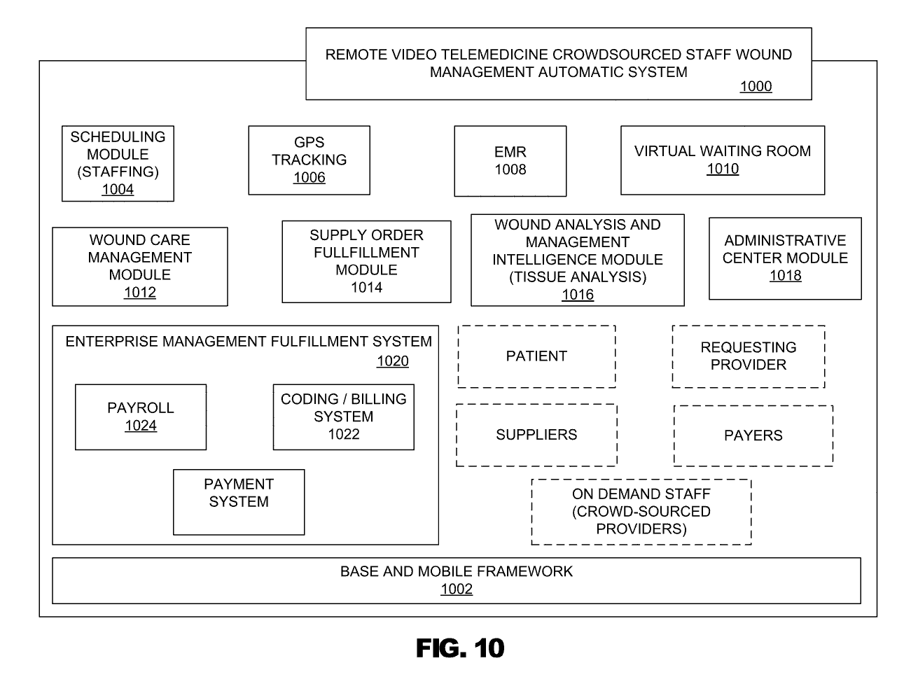

FIG. 10 is a simplified block diagram of a wound management system 1000 illustrating representative modules, according to some embodiments. The system 1000 includes an underlying base and mobile framework module 1002 and a care team (including a telemedicine provider and on-location wound management provider) for various functional modules. While all of the representative modules may be included in certain embodiments, some modules may be omitted in alternative embodiments, depending on the particular functionality to be provided by the on-location telemedicine supported wound management system 1000.

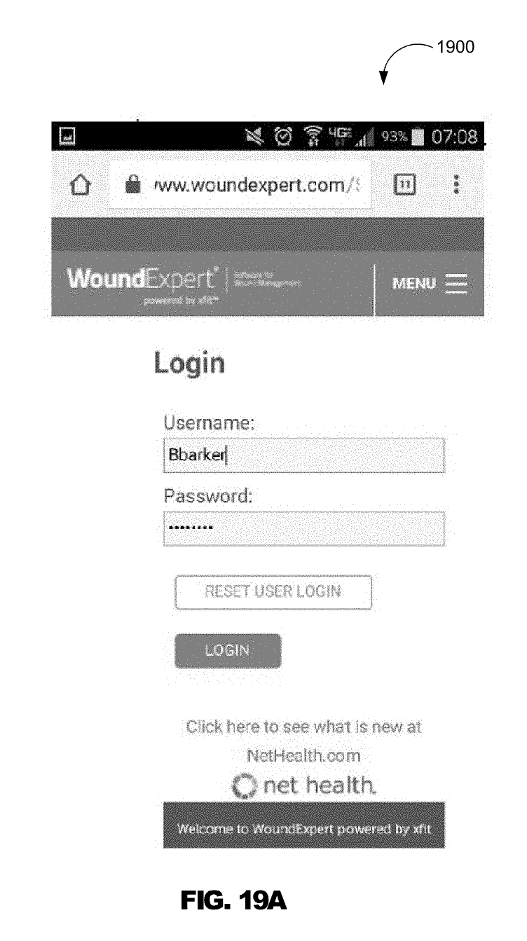

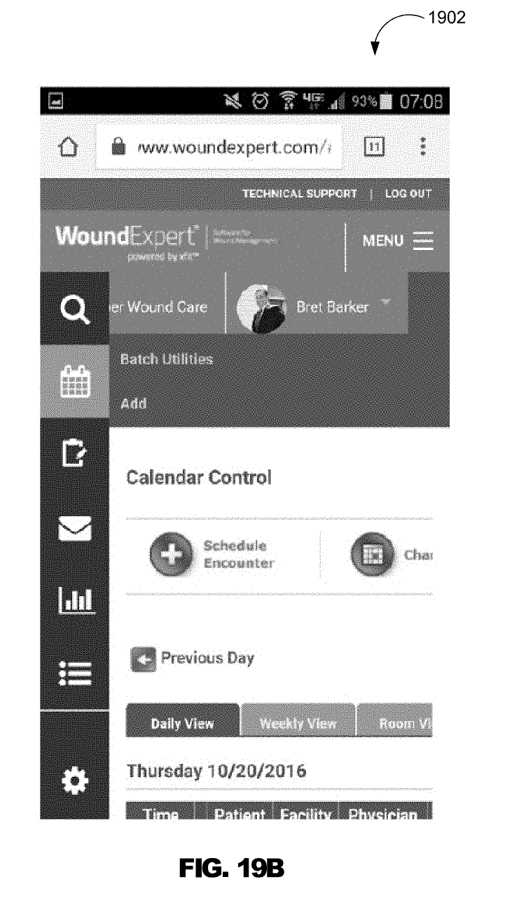

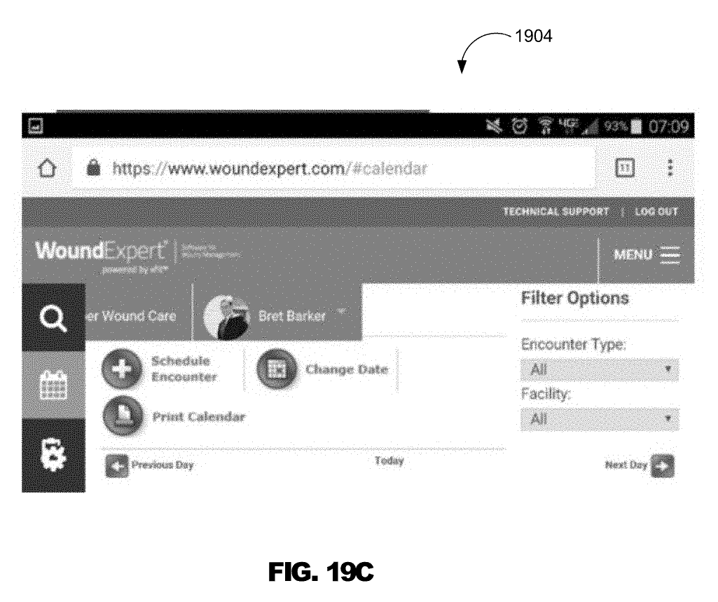

The base and mobile framework 1002 provides a platform for the on-location telemedicine supported wound management system 1000. The base and mobile framework 1002 allows the other modules to operate on the provided platform. Further, the wound management system 1000 includes a scheduling module 1004 that enables the on-location office and licensed crowd sourced providers to dynamically create, publish, and synchronize schedules bi-directionally in real-time, while licensed crowd sourced providers are in the field. This is depicted in FIGS. 19A-C. Yet further, the wound management system 1000 includes a GPS tracking and travel management module 1006 to obtain worker and patient location information. Further, the GPS tracking and travel management module 1006 may dynamically generate route based on schedule and traffic. The GPS tracking and travel management module 1006 may use map database such as those offered by Google Maps™, INRIX™, NAVTEQ™, and Tele Atlas™.

Further, the wound management system 1000 includes an Electronic Visit Record and Care Plan Module (EMR) 1008. The electronic medical wound care management record and care plan module 1008 may be configured to electronically create, store, communicate, and monitor information on patients, visits, tasks, care plans, and other home-care-related information.

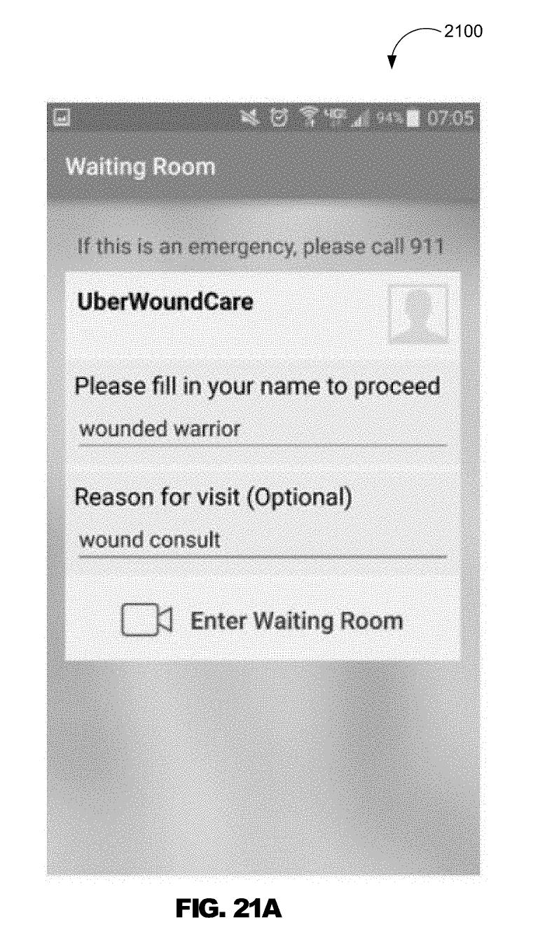

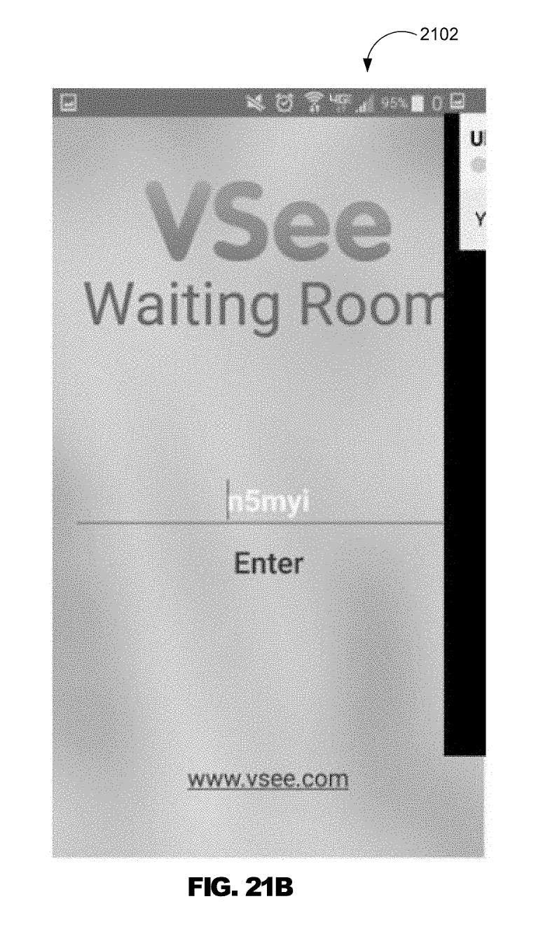

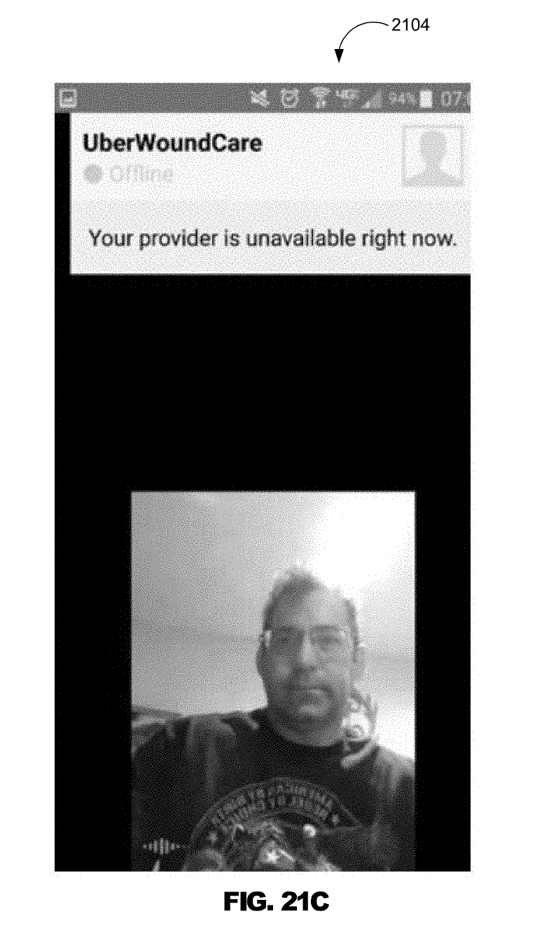

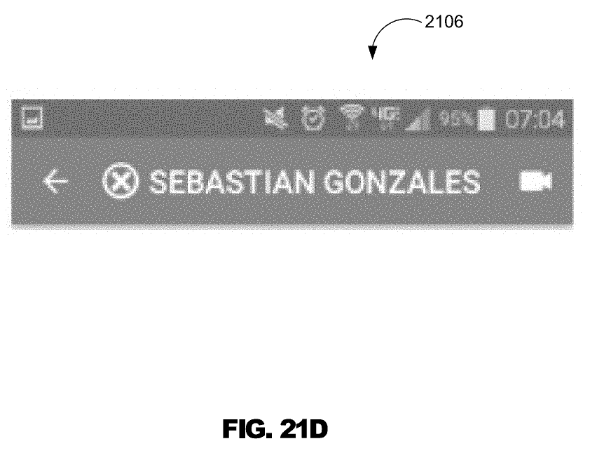

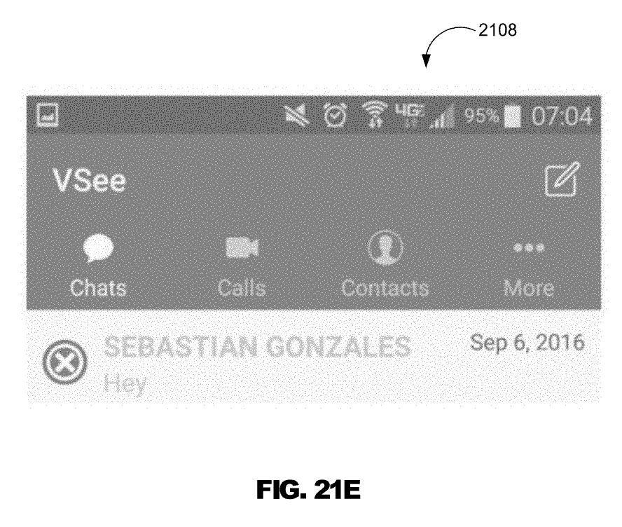

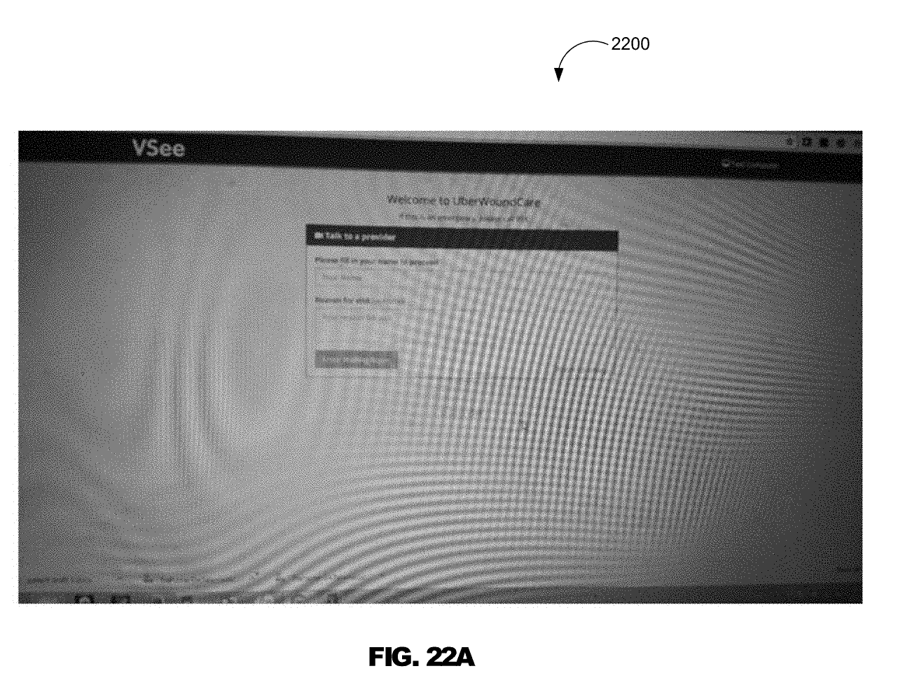

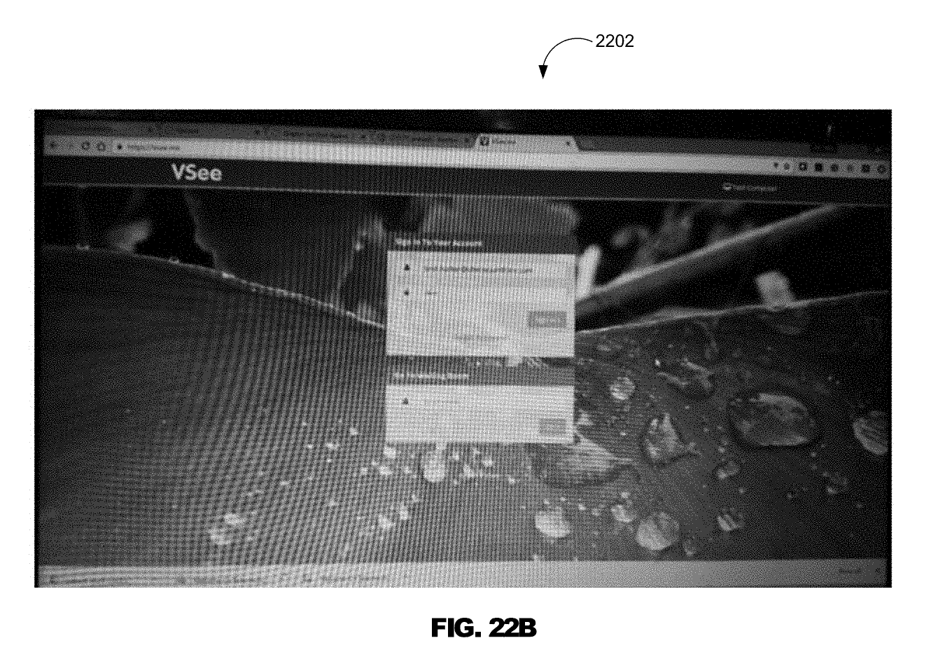

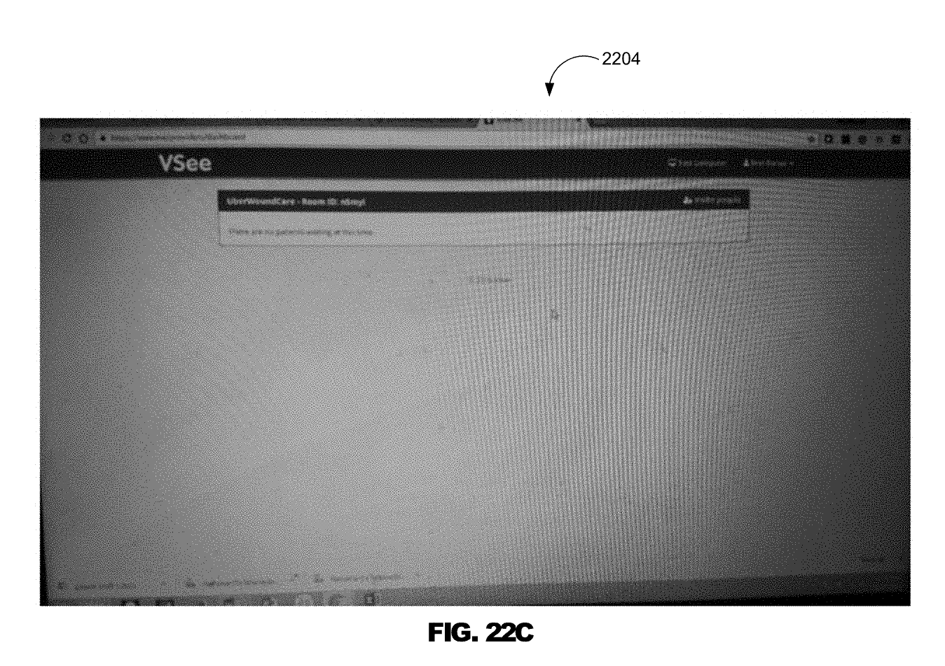

Yet further, the wound management system 1000 includes a Virtual Waiting Room and Video Messaging System 1010 that allows for real-time encrypted mobile too mobile, or mobile to desktop video communications. The communication may be initiated from the home office though primarily requested by the patient for intake and follow up assessments by the remote provider, along with announcements and other purposes. They may also be initiated from client/customer or provider's mobile device, then related to others via the mobile application and cloud based server. For example, an SMS may notify a patient or a provider to check in on the mobile application, providing a link to do so with limited data (e.g. your shipment was delivered at 00:00 PM on 00/00/0000, or reminder your appointment with us is tomorrow at 00:00 AM). Additionally, privacy compliant messaging within the virtual waiting room video and messaging system 1010 allowing for text chat. The virtual waiting room video and messaging system 1010 may use third party applications like VSee™, as depicted in FIGS. 21A-E and FIGS. 22A-C.

Further, the wound management system 1000 includes a wound care management module 1012 that may be configured to capture photos of wounds for remote analysis. Further, the wound management system 1000 includes a supply order fulfillment module 1014. The supply order fulfillment module 1014 may be integrated with the ordering provider mobile application, fulfillment vendor(s), and educational materials tied to the specific supplies shipped to the client/customer/patient's location. Providers may also elect to order supplies directly as needed, as may be the case in agency based staff. The supply order fulfillment module 1014 may utilize stock levels, forecasts based on care-planned items to identify potential need for a specific item for wound management (e.g. Enluxtra™ 4″×4″, 2 each per dressing change, for 4 weeks, then Enluxtra™ 4″×4″ qty. 1 each per dressing change for 2 weeks as the wound size decreases). This allows master fulfillment orders to occur managing inventory prudently while ensuring product availability and reduction in out of stock situations. Alternate vendors for all items and back up supplies may be available, especially useful in metropolitan areas where Amazon™ lockers may be utilized in the case of using Amazon Fulfillment services for non-prescription supplies.

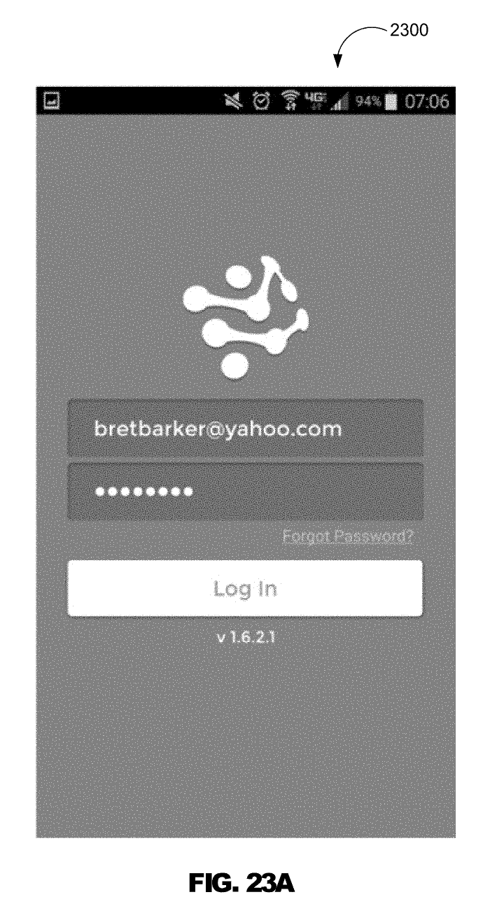

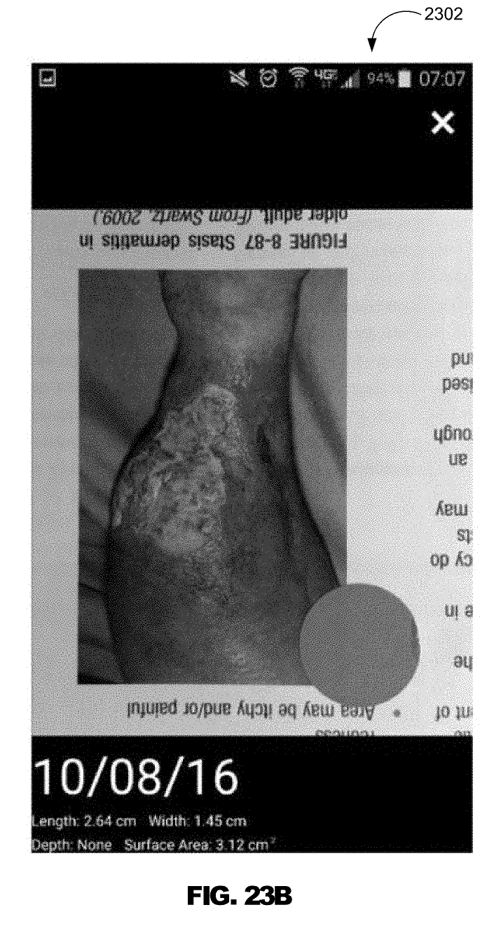

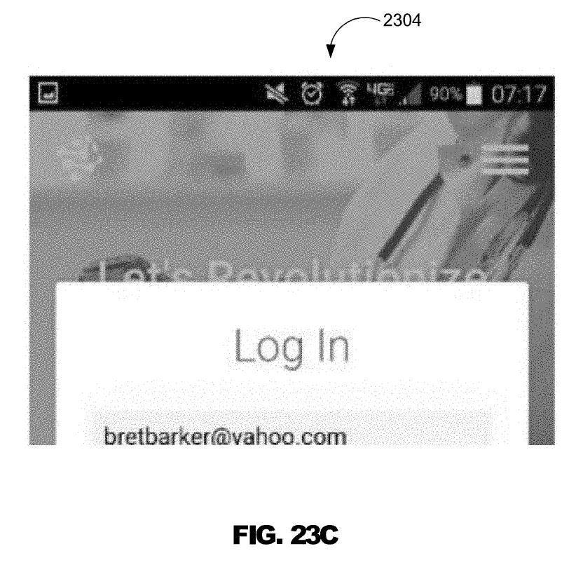

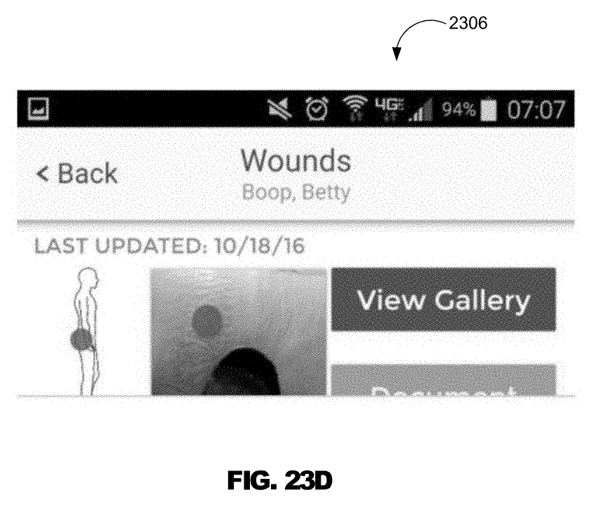

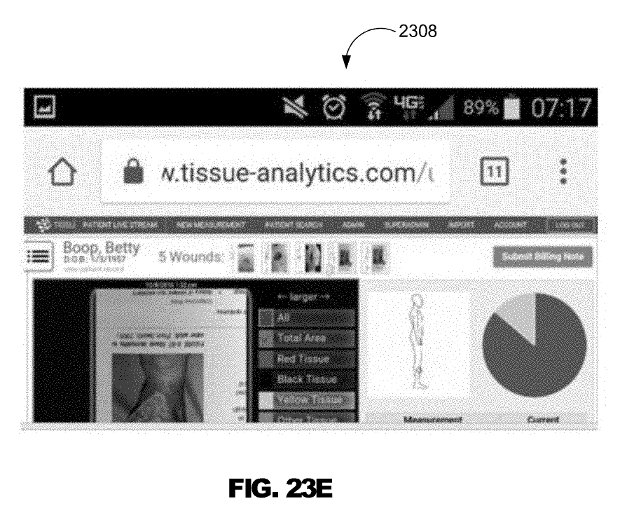

Further, the wound management system 1000 includes a wound analysis and management intelligence module 1016. The wound analysis and management intelligence module 1016 may provide licensed crowd sourced providers with information and reminders regarding their patients' wounds, mapping tissue types, wound size, calculate reductions or increases in sizes and types of tissues which is useful for identifying wound management goals, dressing types, supplies needed, time required for care, and cost determination. Once care is initiated and the first digital photographs are recorded in the system, the ordering provider reviews the data and takes appropriate action. This is depicted in FIGS. 23A-E.

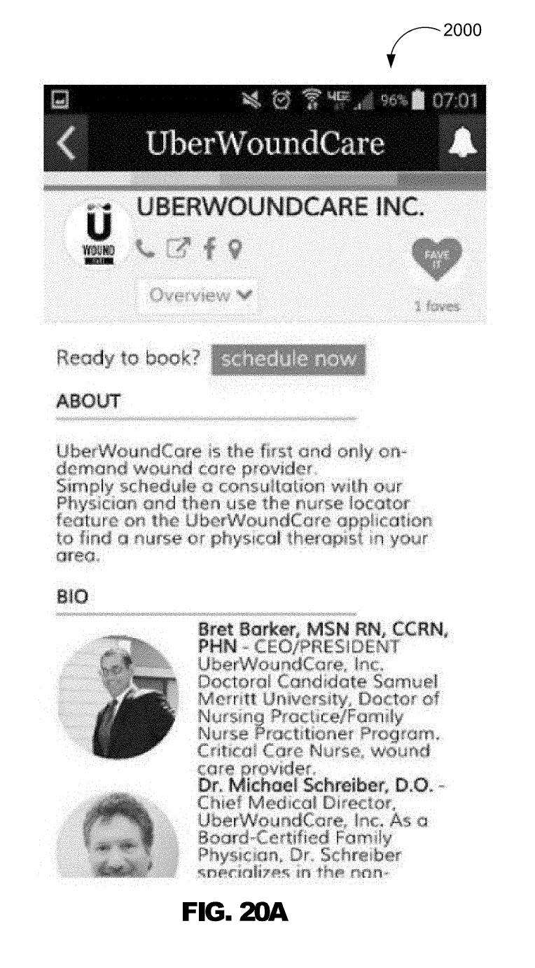

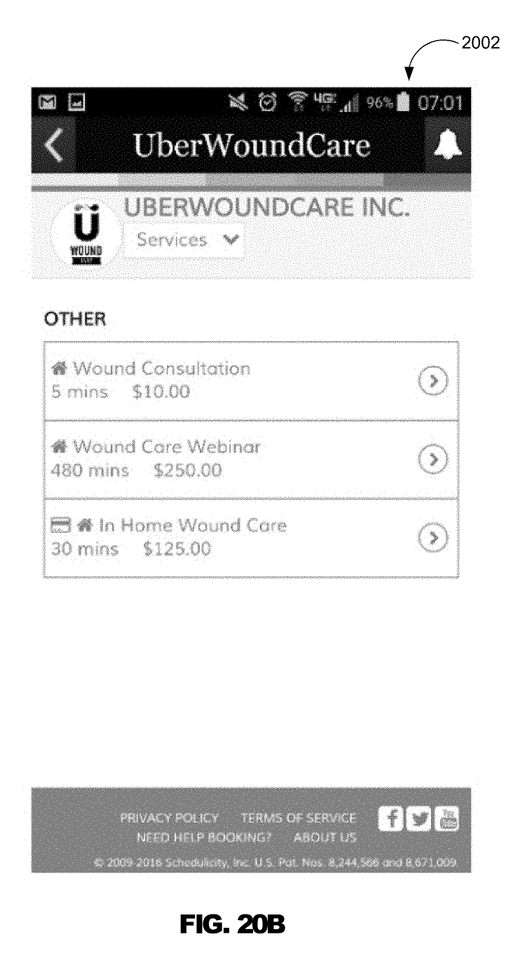

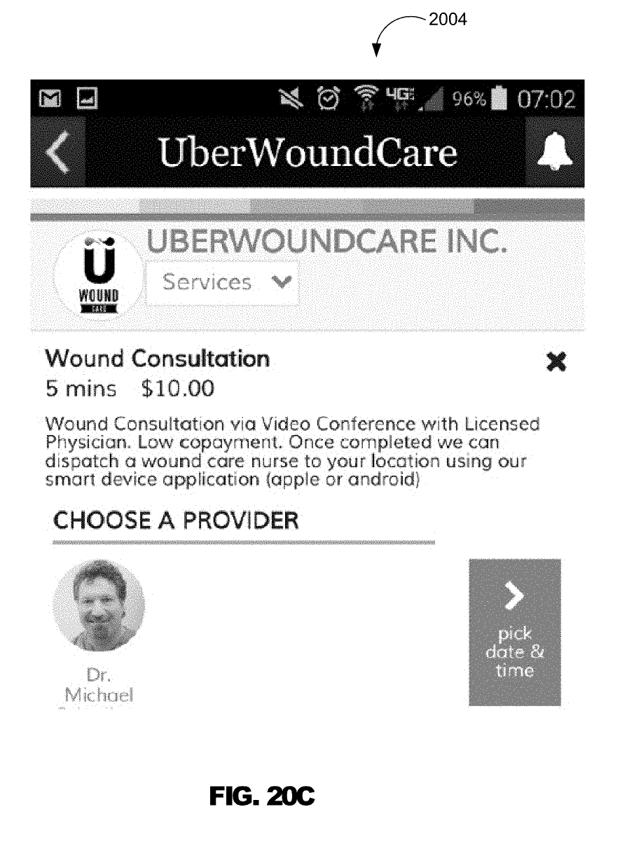

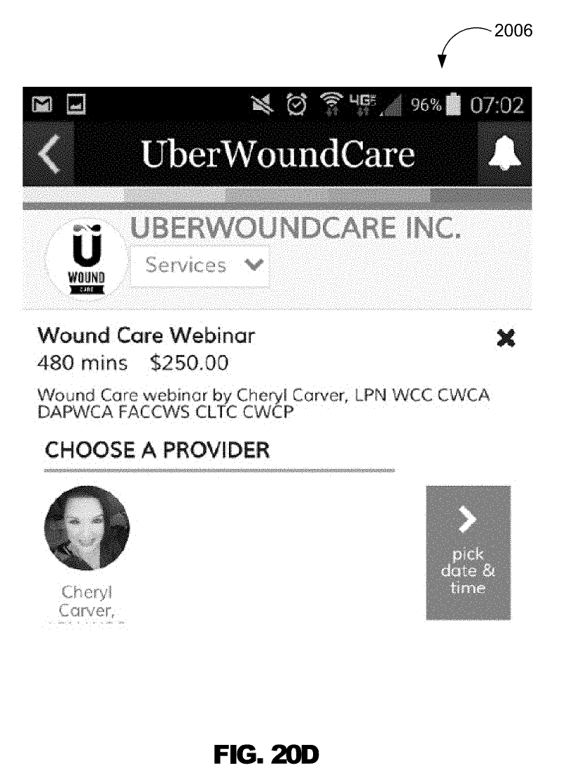

Follow up items may be prescheduled, including future appointments, requesting client/customer to upload photographs or other information. The mobile application and cloud based server may maintain a database of clinical contents along with such rules in order to send out messages and/or alerts (e.g. through SMS messages to the mobile device with link to privacy enabled application), based on data sent by the licensed crowd sourced providers from the mobile device to the mobile application and cloud based server. For example, a client/customer, upon transmitting a current digital wound photo from the mobile device to the mobile application and cloud based server, might receive a message on the server computer indicating that the measurements require a change in care and a consultation with a provider (nurse, nurse practitioner, physical therapist, physician, physician assistant) may be needed. This is depicted in FIGS. 20A-D.

The ranges of either increase size of the wound an or increase in less viable tissue (slough, necrotic tissue, stuck wounds, etc.) may remind the patient to perform recommended dressing changes and discuss the plan of care if changes are needed, in order to assist in reducing the patient's wound size and lead to wound closure (healing).

Further, the reports generated from the uploaded digital photos may be e-mailed or digitally sent directly to the EMR module 1008 for attachment to the patients' medical record requiring review on receipt within a specified time period to ensure appropriate on-location wound management occurs.

Further, the wound management system 1000 may include an administrative center module 1018 which may be configured to manage users, manage visits and tasks that may be performed by a licensed crowd sourced provider for a particular function.

Cases are started after referral (self or from physician), and patient information initially entered into the scheduling application, credit card information stored for co-payment processing. A medical record number is assigned, appropriate documents signed regarding patients' rights, privacy, release of information, and de-identified health information are provided. The patient enters private health information, demographics into the electronic medical record (EMR) patient portal on the cloud based server. At this time the patient may schedule a video face to face wound consultation with wound viewing by a remote provider. After the video interview, an access code may be given and instructions on photographing wounds may be given and appropriate materials needed for local purchase to utilize the program. Simple images may be loaded to the cloud based server and not run through tissue analytics by the patient/client/customer also, and formal tissue analysis may then be performed by the on-location on demand licensed provider. Follow up photographs may then be loaded to the system by the client/patient/customer/caregivers and during subsequent visits.

The on-location business operating the on-location telemedicine supported wound management system 1000 may provide a web-based portal offering anytime/anywhere information access, to that the business may be managed virtually. This promotes telecommuting and may generally tend to reduce timelines associated with scheduling, approving, and submitting invoices for payment. This, in turn, may shorten accounts-receivables timelines, which may typically be a financial benefit for the on-location business. The wound management system 1000 may have a single server computer to act as a web portal, or there may be multiple server computers at a single location (for load balancing and redundancy, for example), or there may alternatively be different server computer affiliated with regional or local on-location offices and having different web addresses from one another as is allowed by logon capability and lack of restrictions at the cloud based server provider. Hardware and software firewalls may be used to prevent outside United States access preventing providers from contacting patients while travelling, so alternate providers may be used unless specific cellular and internet privacy additional security measures may be installed for those cases (e.g. physician travelling in Italy, patient wants to speak with usual wound care provider, case by case access through local internet accomplished, antimalware, antivirus, and other protection software analyze any and all data prior to transmitting separately to the cloud base server). Further, it may not be possible to access the wound management system 1000 from outside the United States. Additional countries utilizing the service may have similar protocols and facilities utilized to prevent loss of information or security breaches.

In an embodiment, visits may be managed through a virtual waiting room, connected via cloud based server allowing video conferencing with patients, access to the electronic medical record, triggers from orders by providers to directly transmit to fulfillment module 1014 to ship wound management supplies to the patient's location either (distributor, manufacturer, wholesaler, Amazon™ services, or other fulfillment method).

After the appropriate patient information is entered in the administrative center module 1018, the provider creates or edits a patient template by wound type(s) and a task list by checking which tasks an on-location on demand licensed staff is to perform during visits which is automatically stored. This creates an open visit allowing assignment of a local provider (agency staff) or ability to be selected from crowd sourced staff listing (both agency staff and crowd sourced staff are listed on the provider database) allowing patient preference. Alternatively, with low staff levels in certain areas, assignment of a licensed on-location on demand provider may be made and the appointment scheduled. The open visit is identified and typical treatment times identified and a two-hour window of service is give. Protocol is to have the on-location staff at the early part of the two-hour window (e.g. 8 am to 10 am is the window, with 8 am preferred arrival time). Post provider video conference, the assigned on-location provider may contact the client/patient/customer and confirm appointment, via secure video chat or secure text chat as show in the figures using Vsee™ or Chiron health™ or other telemedicine platform just as a provider may use the system. This allows for the patient/client/customer and providers (physicians, physician assistants, nurse practitioners) and licensed staff (nurses, physical therapists) to have secure remote discussions with patients whenever they schedule them, even with short length (e.g. 5 minutes or less) to ensure regular follow up, wound assessment management, education, answering of questions and other forms of support to enhance quality of life for the patient/customer/client.

An open visit is a visit that is being performed by a licensed crowd sourced provider in the field as previously discussed. Visits may thus be monitored as they happen by GPS logon on site and check out. Licensed crowd sourced providers perform visits using the mobile devices. The administrative staff is able to view visits that have been started by a licensed crowd sourced provider in real time.

Further, the wound management system 1000 includes an Enterprise Management and Fulfillment System 1020. The enterprise management and fulfillment system 1020 may be integrated with applications from third parties. The enterprise management and fulfillment system 1020 includes application components that are designed to communicate with other on-location systems and has features to support multiple communication protocols, including, without limitation, VPN, HTTPS, HTTP, FTP, and Secure FTP. Possible data structures that may be embodied in such communication protocols include PDF, HL7, XML, CSV, and other formats. The enterprise management and fulfillment system 1020 is flexible to support real-time communication and file batch communication. The enterprise management and fulfillment system 1020 also includes a data mapping utility that maps incoming data messages from the third party format into its own data format that the database server supports.

For example, the coding/billing/accounting system 1022 may be a third-party system. The enterprise management and fulfillment system 1020 may allow that third-party application to integrate and interoperate with applications on the cloud based server, for example. This may enable administrators and/or other home office staff to view integrated and complementary views of financial and coding/billing information. Other third-party applications that might be partly or entirely integrated into the system using the enterprise management and fulfillment system are a scheduling and/or payroll system 1024, a clinical medicine database application, Medicare compliance applications, and others. The information exchanged between the enterprise management and fulfillment system 1020 and third-party applications may be exchanged in real-time or near real-time, for example. Predetermined ICD-10, CPT and other codes may be associated with care episode types, materials, and providers to perform this near real time task to improve data integrity, reduce denials, and lessen burden on providers to float cash flow waiting on payments by coding through a rules engine to detect errors and correct them with a live person overview prior to submission. Data may be stored in the case for periodic payments as with worker's compensation cases maintaining same data integrity and billing capabilities.

A part of the processing related to the system is software which is targeted to mobile devices (i.e., the “client”). When an on-location on demand licensed staff, such as a provider, enters a record whether at an office, home or other location, the on-location on demand licensed staff launches this specified application. Both client/patient/customers and providers/licensed staff may be utilizing mobile devices remotely and in the field, while some providers and licensed staff may utilize laptop/desktop access devices for optimal viewing depending on need. Ideally every person involved may have the ability to utilize the system 1000 only with mobile devices (based on preference and technical abilities of the users).

A logon must be created for the on-location ordering of services and payment data entered, and to order video conference the customer/client/patient is first prompted to enter characters identifying oneself.

With this data, the client software attempts to make contact with the cloud based server, and a patient medical record is created via an encrypted communication protocol.

Further, data is entered into the system 1000 by the patient/client/customer on the electronic medical record in the patient information area as given access. The provider when assigned may enter pertinent information as required during discussion, follow up and treatment. Upon successful communication between the client mobile device scheduling module and creation of appointment, on mobile device and server computer, the server computer is able to provide the client mobile device with the tasks that are associated with the patient. This indicates the creation of a “pending visit,” and shows up on the waiting room list for the provider to video conference with the client/patient/customer along with tasks that are associated.

If communication is not established between the server computer and client mobile device, the client mobile device may move into a “provider unavailable” mode. Within a few minutes of the appointment, a pending visit and notification may be flashed. Another example of unavailable provider may be greyed out screen icon, when a provider wants to remain in the wound management system 1000, but has limited available vacation.

On-location providers may enter data into the wound management system 1000. All information and templates are typical of recording data required by payers (Medicare/Medicaid, private insurers) and the data is presented in different forms graphically depending on device used (mobile or desktop/laptop).

Examples may be a numerical number to indicate: the patient's wound photograph, type of dressings used, vital signs (temperature, the patient's pulse rate, etc.) and associated data. The application on the client mobile device provides an interface which collects this data from in the user/on-location on demand licensed staff as seen. Red check marks may indicate loaded data locally and not uploaded due to lack of internet connectivity which is less a hazard now than in the past, and data may be uploaded immediately once connectivity available, and no local device storage may occur.

When the on-location on demand licensed staff enters required data, the client application validates the data and automatically transmits the data. Each screen contains templates with required data to be entered prior to moving to the next step. This data is cached and once all is entered and transmitted and accepted by the cloud based server, the next data screen may then be able to be accessed (e.g. enter wound type as pressure injury, no stage given, cannot go to next screen). The data is continuously transmitted via cloud based server to run tests against rules on the templates the on demand on-location licensed staff or provider is entering to ensure complete recordkeeping reducing denials of payment and assuring patient database is sufficient to record the wound status.

If connect attempt fails to reach the server computer, the application on the mobile device may store the data associated with the visit onto the data repository of the mobile device as cache. This data may be held until such time that the mobile device makes a successful connection with the cloud based server. A connectivity alert may be triggered in loss of connection status indicating a “downtime” or paper documentation may connect, if the local on-location provider is unable to troubleshoot connectivity issues

After scheduling an appointment and scheduling a wound management visit as discussed, if no provider is assigned by the local agency or patient has not chosen a local crowd sourced licensed wound management, then an office administrator (admin) refers to employees at the local office (Location) level, responsible for setting the daily tasks for the field personnel on-location on demand licensed staff (such as a home healthcare service staff member or licensed crowd sourced providers).

At the local agency level, including Home Health Agency utilizing the system 1000, primary care providers, discharge planners, outpatient or hospital based wound clinics and free standing wound clinics or urgent care centers may access the data and manage the patients care outside of the specific wound care items provided by the service in the telemedicine remote providers and crowd sourced staff.

This includes creating, reviewing and editing task lists that the on-location on demand licensed staff is to perform during a visit to the home of the patient(s). Visits are monitored as they happen (Open Visits). This further entails reviewing and editing, if necessary, visits that have been completed by the on-location on demand licensed staff (Pending Visits) and approving visits (Approved Visit).

Alternatively, the cloud based server application provides a method to import patient information using the following methods, without limitation: Remote using VPN, via SFTP Remote via HTTPS Local via TCP/IP Formats, CSV, XLS, DBF, XML, or database connection. Preferable use of information transfer is through API (application programming interface) to transfer data easily to various systems such as Epic™, Meditech™, and others with a basis of data transfer of reports as a portable document format (PDF) which are readily readable and searchable.

Open Visits are those which have yet to be performed, or a “que”. This shows patient demand, need for providers and is reportable by zip code, latitude and longitude to aid in identifying staffing needs. Further reporting of this information as de-identified may provide public and government agencies with data identifying higher problem areas along with data regarding payment type.

Pending Visits occur when an on-location on demand licensed staff completes a visit, the administrative staff is able to view the on-location on demand licensed staffs finished visit in the Pending Visit section of the cloud based server application.

Visit approval, is determined with payers based on rules and treatment type ordered by the provider, so that a determination is made as to whether the visit is approved or needs editing. The administrative staff is able to determine if a visit is completed properly using “Pending Visit”. If the Visit is complete, the visit may be moved to next stage. Visits may be edited for compliance with coding and billing requirements of payers and must be in a “completed” status after editing prior to sending through the billing and coding engine provider. If the visit is not complete, it may be edited for completeness and then moved to the next stage, “Approved”. Once a user moves a visit to this stage, the visit may no longer be edited or deleted only an addendum note may be added at a later time (e.g. on-location staff determine tunneling present which did not appear during video conference with remote provider although patient was asked about the issue).

Visits approved and visits archived are those identified that are completed and may be stored and not appear on a listing to reduce clutter and make viewing of pending and open visits easier.

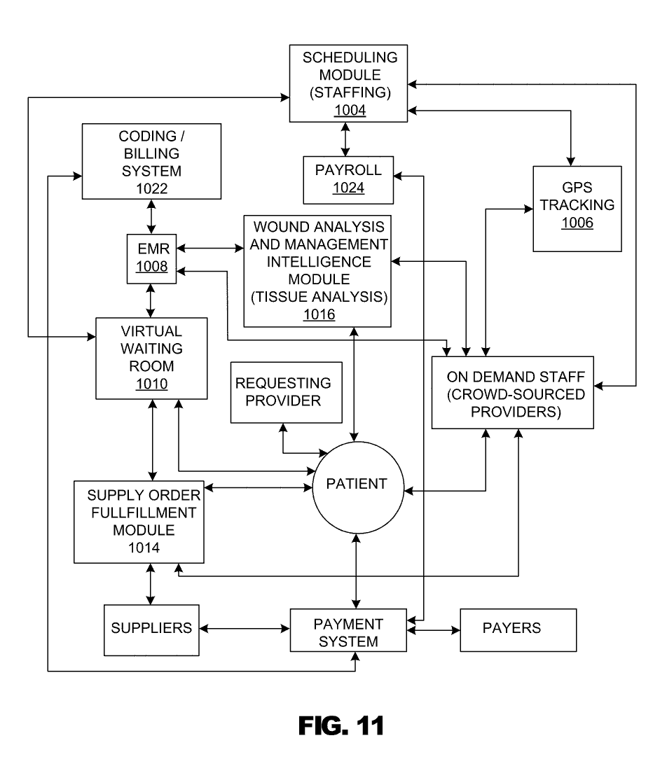

FIG. 11 is a block diagram of a wound management system 1000 illustrating communication among various modules, according to some embodiments. As discussed above, one or more mobile devices that may be used by a home-care staff person and/or licensed crowd sourced providers to cloud based video communicate with base/office mobile application and cloud based server. The bidirectional communications are preferably over the Cellular data, Wi-Fi to connect internet, which the mobile device may access through a gateway. The preferred protocol used to communicate data is HTTPS (Hypertext Transfer Protocol with SSL (Secure Socket Layer) encryption for security) with additional encryption within each of the mobile application modules, with hardwired lock at cloud server provider location (data center) at top of the application stack entryway locking out all other countries except the United States at this time. Additional counties may require integrated secure data centers at their locations and may not make possible a worldwide integration due to political volatilities. At this time protection of protected health information within a patient's respective country is essential. Protocols for accessing data and transmission as needed may occur on a case-by-case as a patient travels and support may be needed out of the country or in international waters.

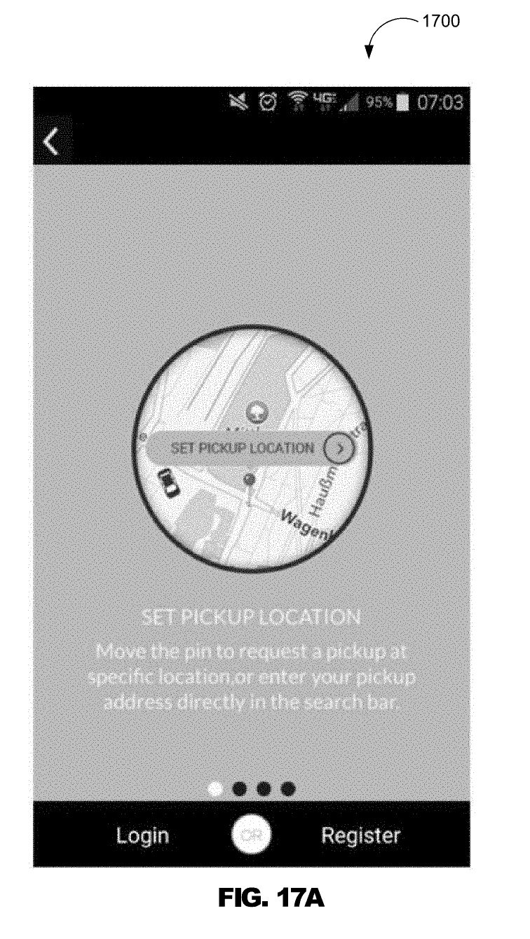

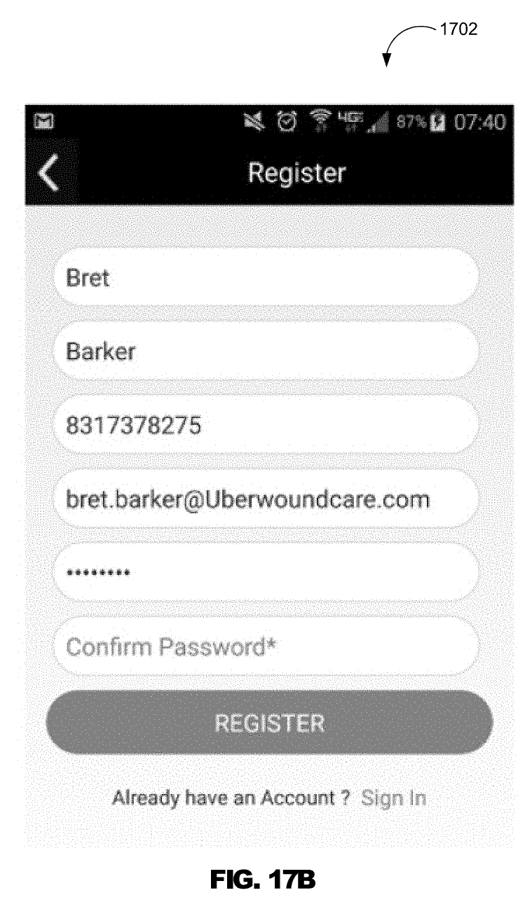

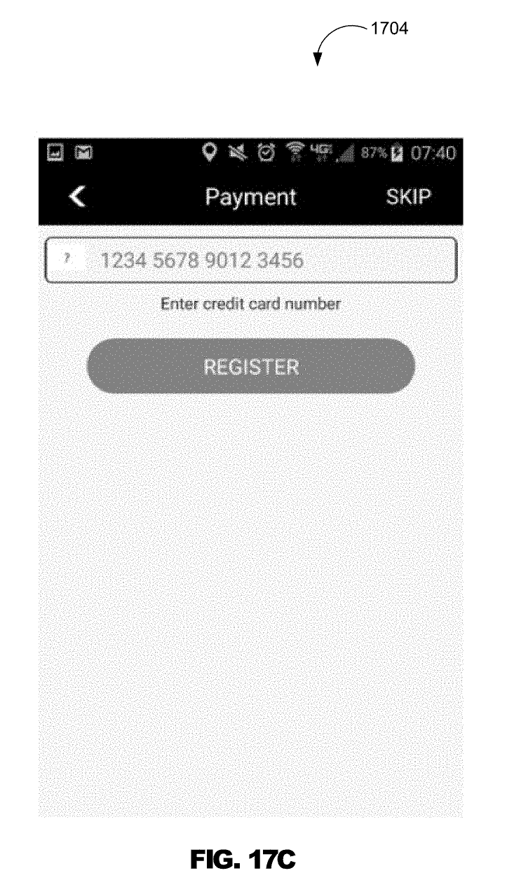

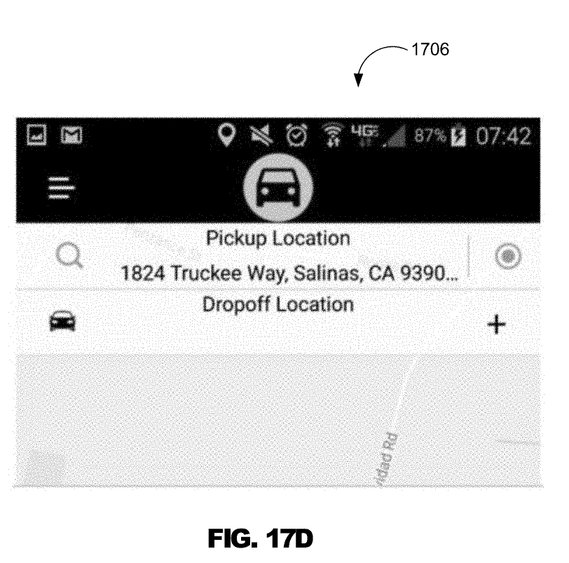

The visiting staff may select the patient visit number from the scheduled list on the mobile application verifying GPS location which automatically time and date stamps the visit into the medical record upon clicking the check in or pickup case button. This is depicted in FIG. 17A-D.

The visit record is automatically saved via the mobile application and cloud based server over a communication network (cellular or Wi-Fi) identifying that a visit is starting for a specific patient. In one example, the mobile application and cloud based server automatically responds to the visit that is identifying the nature of the wound care management episode, prompting digital images to be taken and charting completed the episode of care. Digital wound information may be visualized by the remote provider using the encrypted video messaging system after the patient setups an appointment in the virtual waiting room. Live video feed of the wound allows the remote provider to make an accurate initial assessment to order treatment and develop a plan of care. Once under the systems care, the patient/client/customer may have access to further digital software embedded in the application, requiring a standardized and colored Avery sticker readily available at any store with stationary supplies. This allows for color correction, lighting correction and provides a size reference for the tissue analysis software to compute the data regarding the wound (size, area, tissue type, tissue colors). This tissue analysis software is used by the licensed on-location wound management providers also with more detail entered into the record than a patient may provide. Ability to continually send wound progress data beyond having a provider on-location provides a database of information unparalleled in comparison to intermittent hard photographs placed in paper charts during regular office to visits at bricks/mortar sites. Some bricks and mortar sites do utilize digital photographs sent in by remote providers and sometimes patients, while we provide full tissue analysis, data encryption and protection of private health information.

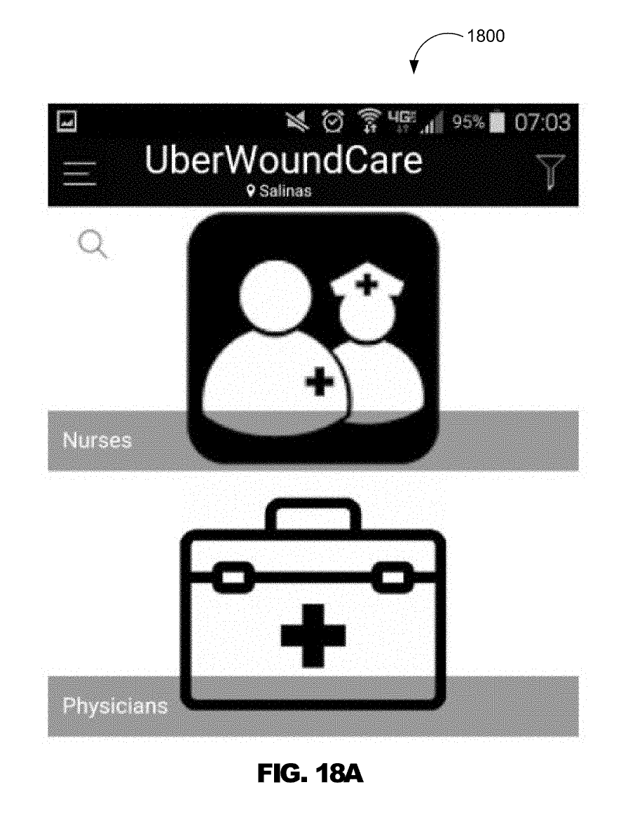

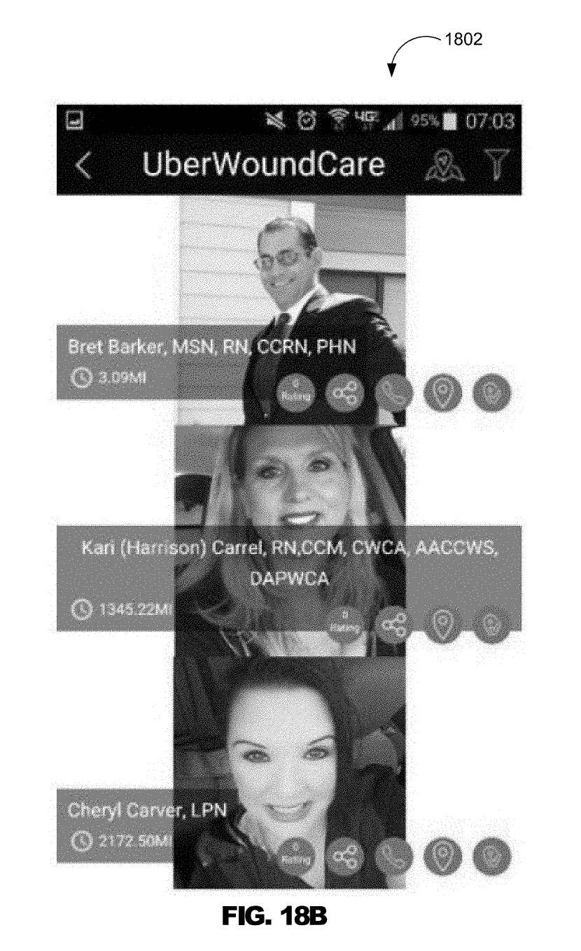

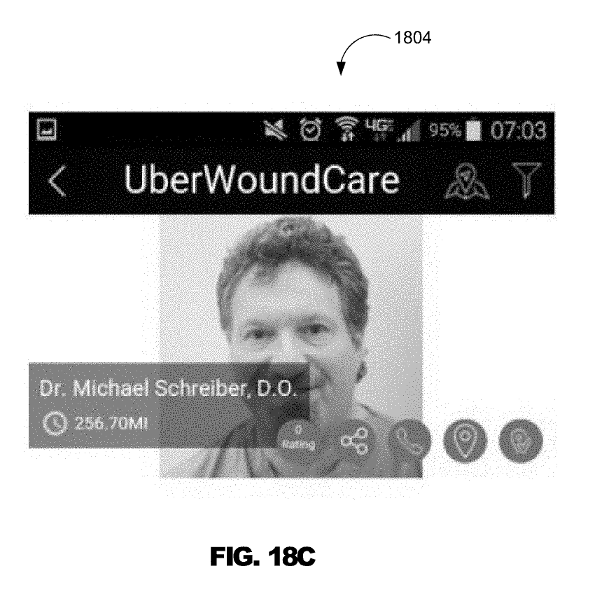

Alternatively, information and wound care instructions may be manually inputted by an administrator and sent to the on demand on-location visiting provider which may appear on the EMR record to do list. This is depicted in FIG. 18A-C.

Communication with a wound expert (nurse, physician, or other provider) may also occur using the mobile application at any time during the visit by the on-location provider. This feature provides face-to-face communication via encrypted video with a physician within minutes. Any additional information or addressing complications which may occur during an episode of wound management thus may be visualized and addressed by a mid-level provider or physician immediately. This allows also for a provider to go to an additional location as needed should that provider still be available after the current visit, such as an alert, may notify staff a patient/client/customer nearby the provider requires care.

By receiving this information from the administrative computer-based mobile application and cloud based server, the on-demand on-location visiting provider knows specifically what the patient needs are, where the patient is located, and estimated travel time from current location, i.e. work, home, school, grocery store) to the patient's location, home, office, hotel, campsite, beach, school ideally in a route towards the on demand on-location visiting provider intended destination. This is to reduce travel outside of the area, minimize time lost, maximize per diem care, and ensure local staff caring for patients in the nearby area. This may be less than 15 minutes out of range. The record indicates what to do for that patient, what supplies were sent to the location, the tracker number for the items, and arrival date, then a patient specific visit record may be generated.

In an embodiment, the system 1000 (via the GPS module 1006) on setup requires the ability for client/customer/patients to identify and select licensed providers in their area, thus on entry a local point of reference such as health agency, post office, grocery store or other commercial location (tracking address) may be located as the home base location of the provider, rather than the licensed providers home. Home addresses may be only loaded in the system for payment and administrative reasons. By having a location of a provider base location and GPS-tracking enabled, the customer/client/patient may determine when the provider may arrive by tracking the provider on a map. This same mapping system is utilized by the provider to go to the client/customer/patient's location, utilizing readily available mapping and navigation systems. A key feature of the system is a database of providers with addresses (tracking address) which any user of the system may be able to determine the base distance from client/customer/patient to the provider. It is within this ability provider may be located by patient/client/customers needing care and provide the ability for the working licensed provider to provide additional services on days off work, after or before regular working hours, or at other times as the client/patient/customers home may be within or on a route traveled by the provider. Ideally this allows for no additional or minimal travel outside the on-location providers typical travel routes (e.g. to and from work, to and from home, to and from school) providing the ability to conduct the stop at the client/customer/patient's location to provide on demand on-location wound assessment and management.

Templates and graphical user interfaces between the mobile application and cloud based server vary and may be updated from time to time. The data entered is that as to be expected by charting expectations, standards, and requirements for patient care episodes, coding and billing as is industry standard and subject to change. Individual steps to charting may happen from time to time and operational procedures may be modified as necessary to achieve tasks.

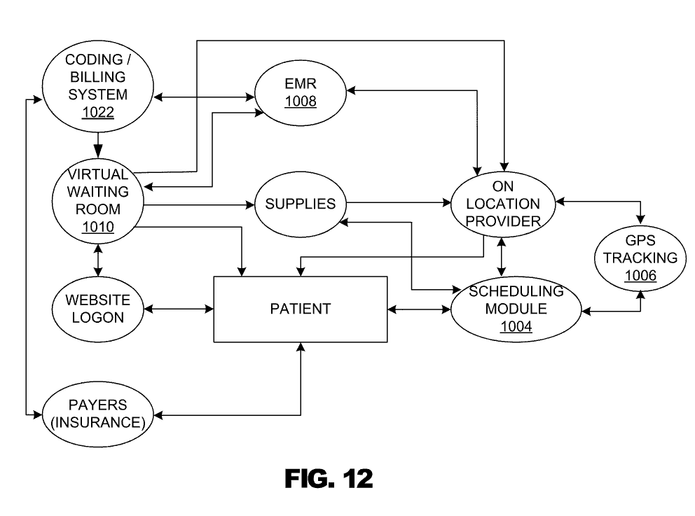

FIG. 12 is a block diagram of the wound management system 1000 illustrating communication among various modules, according to some embodiments. Further, the block diagram depicts relationships between communication devices of field service users, on location on demand licensed staffs, patients, in accordance with some embodiments.

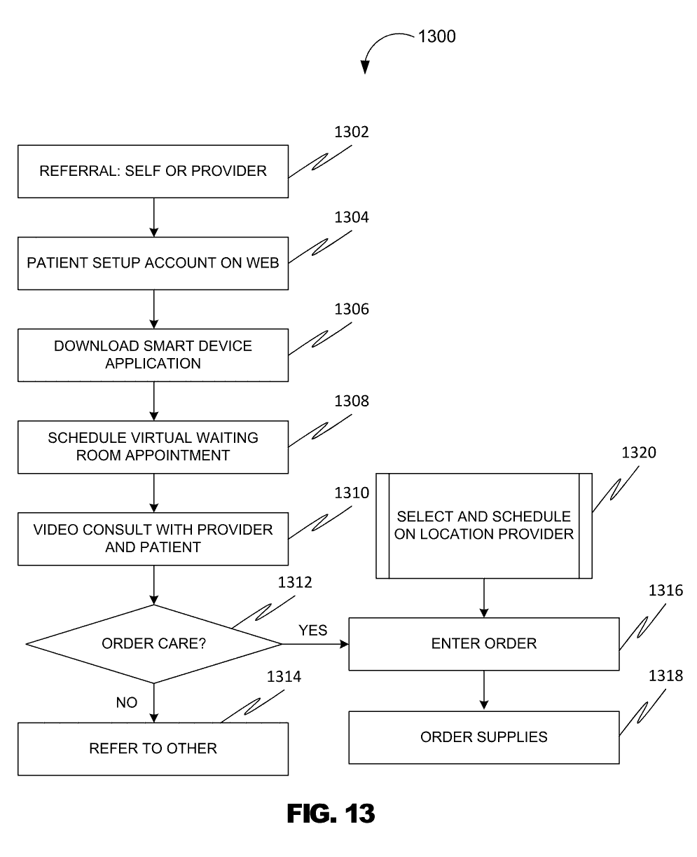

FIG. 13 is a flow chart of a method 1300 for initiating and establishing bi-directional communication with a communication device, a remote mobile application and a cloud based server, in accordance with some embodiments. At 1302, the method 1300 includes referring users. The referral may be self or sent by a provider. Then, at 1304, the method 1300 includes setting up patient account on the web. Next, at 1306, the method 1300 includes downloading a smart device application. Further, at 1308 the method 1300 includes scheduling a virtual waiting room appointment. Yet further, at 1310, the method 1300 includes video consulting with provider and patient. Moreover, at 1312, the method 1300 includes checking if any order has been placed. If no order care is placed, then the patient is referred further at 1314. However, if an order is placed, then, the order is entered, at 1316 and the supplies are ordered at 1318. Further, at 1320, the method 1300 includes selecting and scheduling on a location provider.

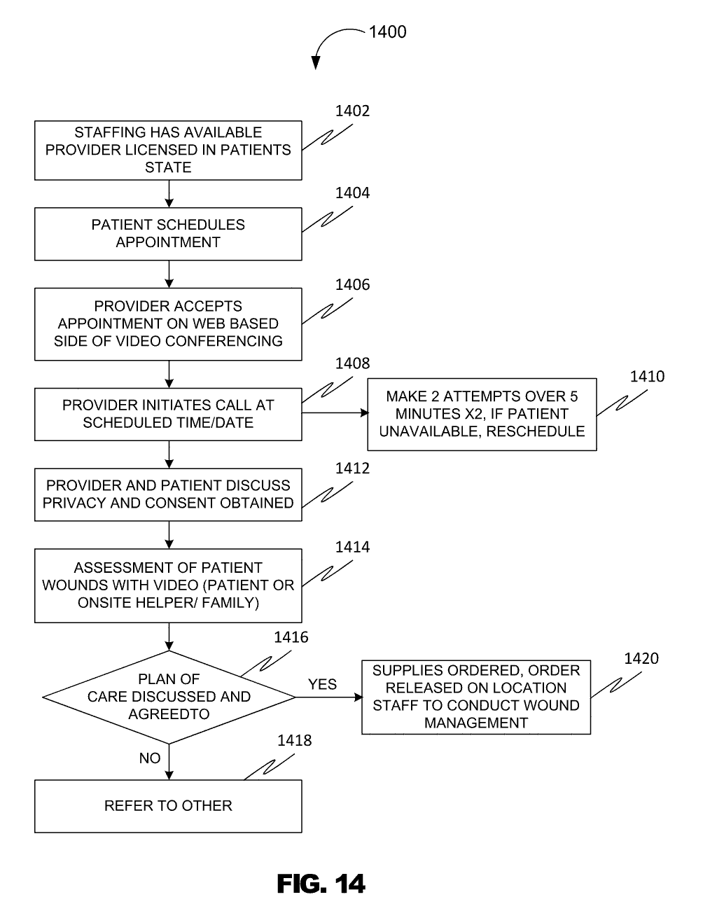

FIG. 14 is a flow chart of an office administrative method 1400 as part of the on location telemedicine supported wound management system 1000, in accordance with some embodiments. At 1402, the method 1400 includes determining licensed providers available in a patient's state. Further, at 1404, the method 1400 includes a patient scheduling an appointment. Yet further, at 1406, the method 1400 includes the provider accepting the appointment on the web based video conferencing. Moreover, at 1408, the method 1400 includes the provider initiating a call at the scheduled time/date. Further, at 1410, the method 1400 includes making 2 attempts over 5 minutes, if patient unavailable.

Further, 1412, the method 1400 includes the provider and the patient discussing privacy and obtaining consent. Then, at 1414, the method 1400 includes performing assessment of patient wounds with video (patient or onsite helper/family). Thereafter, at 1416, the method 1400 includes checking if a plan of care has been discussed and agreed to. Further, if the plan of care has not been discussed and agreed to, then the patient is referred further at 1418. However, if the plan of care has been discussed and agreed to, then the supplies are ordered and the order is released to the location staff to conduct wound management, at 1420.

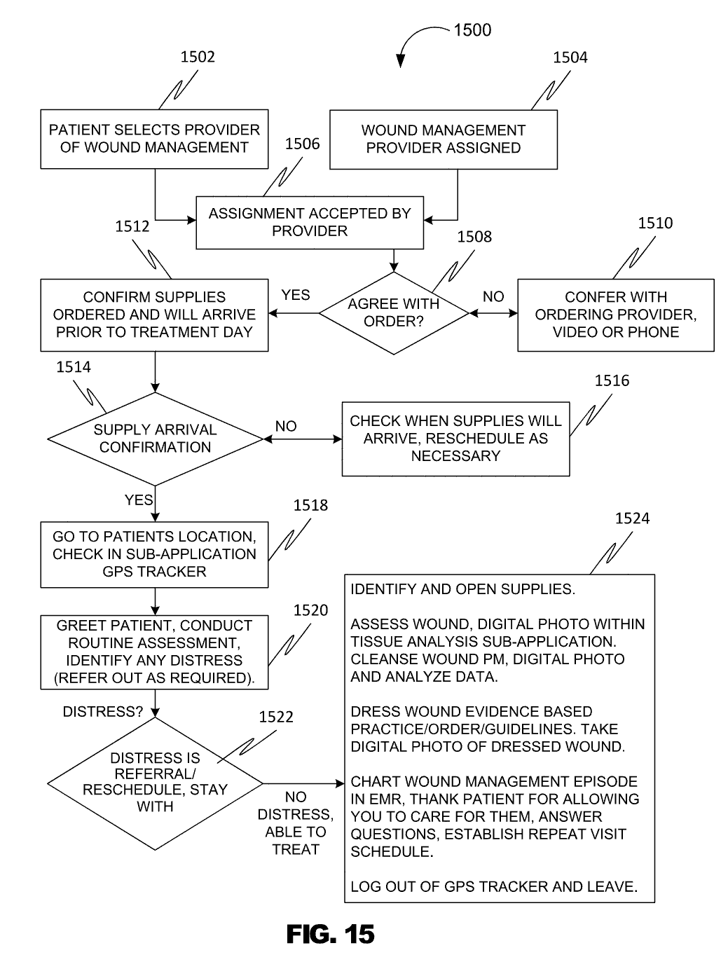

FIG. 15 is a flow chart of a field personnel visitation method 1500 associated with a communication device and related tracking as part of the on location telemedicine supported wound management system 1000, in accordance with some embodiments. At 1502, the method 1500 includes a patient selecting a provider for wound management. Further, at 1504, the method 1500 includes assigning a wound management provider. Yet further, at 1506, the method 1500 includes a provider accepting the assignment. Then, at 1508, the method 1500 includes checking if the provider agrees with an order. If the provider does not agree, then the method 1500 includes conferring with the ordering provider, via video or phone, at 1510. However, if the provider agrees, then the method 1500 includes confirming the ordered supplies and the arrival date (prior to treatment day), at 1512. Then, at 1514, the method 1500 includes confirming if the supplies have arrived. If the supplies have not arrived, then at 1516, the method 1500 includes checking when supplies will arrive, and reschedule as necessary. However, if the supplies have arrived, then, at 1518, the method 1500 includes the provider going to the patient location and checking in sub-application GPS tracker. Moreover, at 1520, the method 1500 includes the provider greeting the patient and conducting routine assessment. Thereafter, at 1522, the method 1500 includes identifying any distress. If a distress is determined, then the patient may be referred out as required. However, if no distress is determined, then, at 1524, the method 1500 includes identifying and opening supplies. Thereafter, the wound is assessed, a digital photo is provided to a tissue analysis sub-application. Further, the wound is cleansed. Further, the wound is dressed based on evidence based practice/order/guidelines. Yet further, a digital photo of dressed wound is clicked. Moreover, the wound management episode is charted in the EMR. Finally, the provider thanks the patient for allowing then to care for them, answer any questions, establish a repeat visit schedule. Finally, the provider logs out of GPS tracker and leaves.

According to further aspects, the method 1500 discloses steps related to a field service on-location on demand licensed staff (e.g. a licensed crowd sourced providers) using the mobile device during the process of a field visit, according to one embodiment, are shown. The field service on-location on demand licensed staff interacts with the mobile device and initiates a first visit component associated with the software-based client application of the mobile device. It is assumed the mobile device is in coverage area for telephonic and/or data communication. If the mobile device is not in coverage, then, a manual task list is displayed listing the tasks to be accomplished by the field on-location on demand licensed staff using the mobile device. Processing then proceeds to block, in which the field service on-location on demand licensed staff completes tasks and records results.

If the mobile device is in coverage, then a patient task list is transmitted from the mobile application and cloud based server to the mobile device operated by the on-location on demand licensed staff in the field. The field service on-location on demand licensed staff completes tasks and records results in real-time through the mobile device connected to the cloud based server on the electronic medical record (EMR) application). The on-location on demand licensed staff completes the visit data gathered and obtained is transmitted by mobile device for receipt by the cloud based server computer of the mobile application. Throughout this process, GPS records location check in time, and check out time to provide time of service estimates for payment, future reference, and reporting (e.g. visit expected to take 30 minutes, and visit takes 31 minutes, within a range estimate. If visit exceeds time needed, evaluation of why occurs, and continues to provide updates of the GPS coordinates of the location of the mobile device as is standard, if a provider leaves the site within 100 feet, an alert to the on-location staff may be made to have them click “check out” and complete the visit on the mobile application and transmit the data to cloud based server.

According to some embodiments, a method for an on-location on demand mobile application telemedicine based wound management system 1000 is disclosed. The wound management system 1000 includes a mobile application and cloud based server accessible via a communication network, the mobile application and cloud based server comprising: a staff self-scheduling module for creating a work schedule for a care team (includes telemedicine provider and on-location wound management provider) of licensed staff (nurses, physicians, nurse practitioners, physicians assistants, physical therapists) wherein the work schedule assigns each of the care team (includes telemedicine provider and on-location wound management provider) of licensed crowd sourced providers to a care team (includes telemedicine provider and on-location wound management provider) of visits with a respective care team (includes telemedicine provider and on-location wound management provider) of patients; a tracking and travel management module for determining visit e information for the care team (includes telemedicine provider and on-location wound management provider) of licensed crowd sourced providers, tracking visit times, and determining actual travel distance between visits by licensed crowd sourced providers. Further, at least a portion of the work schedule is created by a staff scheduling module determined by on-location per diem staff availability loaded into the scheduling module by staff and selected at may by patient or assigned by home base. Client/customers are able to log on to the mobile application or web based portal and select an on-location wound management provider based on time and day availability, along with another desirable metrics chosen by the client/customer. Additionally, in the event of reduced staff availability, assignment of a local on demand on-location wound management provider may be made, which is dynamically updated based on the tracked visit times; visit record and care plan module for administering visit records initiated by the licensed crowd sourced providers during the visits and for designing patient-specific, visit-specific care plans comprising wound management tasks to be performed during the visits; and a communication module for cloud based video communicating care plans, scheduling information, and route information to a care team (includes telemedicine provider and on-location wound management provider) of mobile devices via the communication network, wherein the care team (includes telemedicine provider and on-location wound management provider) of mobile devices are associated with the respective care team (includes telemedicine provider and on-location wound management provider) of licensed crowd sourced providers scheduled to visit the respective care team (includes telemedicine provider and on-location wound management provider) of patients, wherein the mobile application and cloud based server transmits the care plans to the mobile devices so that the associated licensed crowd sourced providers receive information regarding tasks to be performed on the patients, and wherein the mobile devices are used by the licensed crowd sourced providers to transmit back to the mobile application and cloud based server visit record information regarding performed tasks.

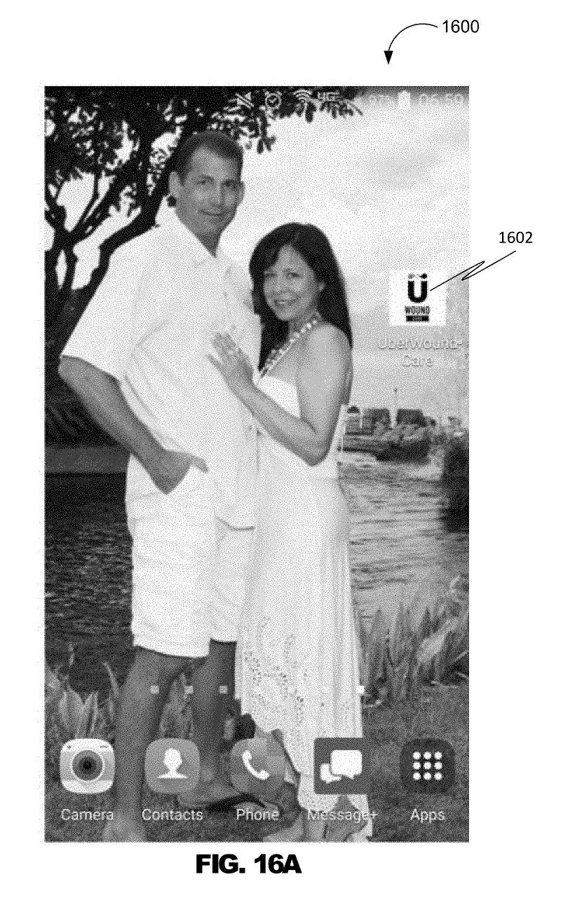

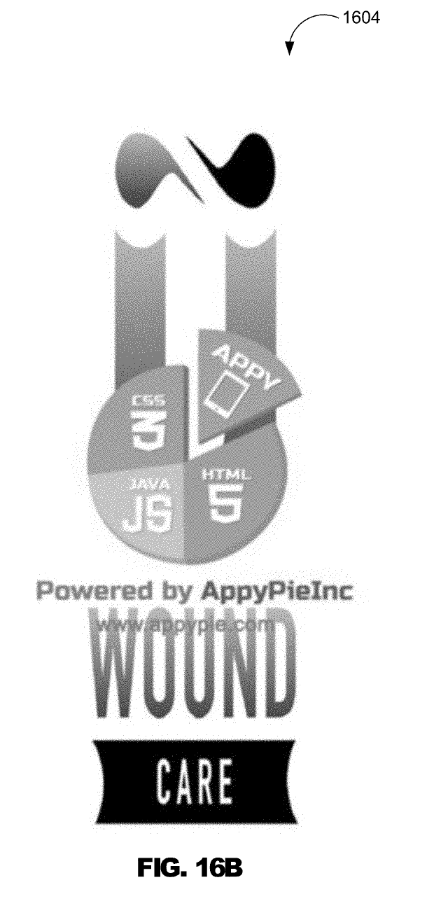

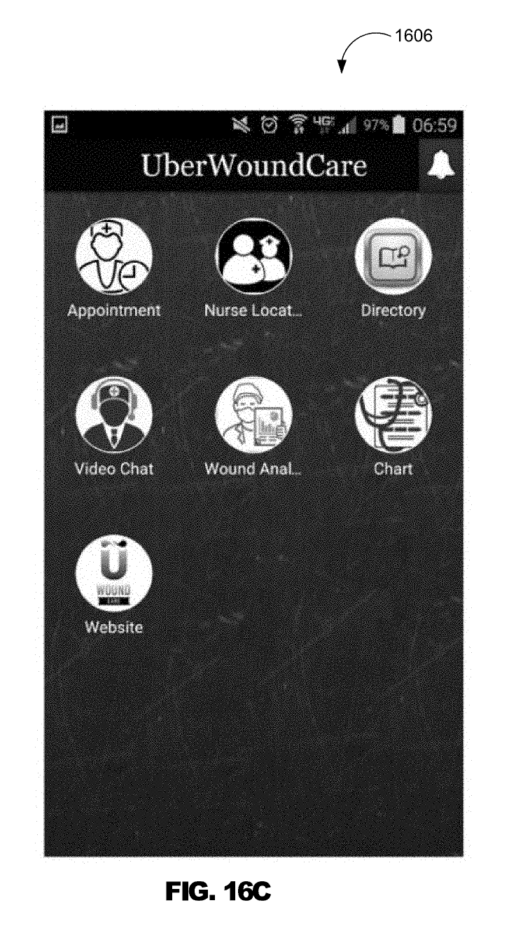

FIGS. 16A-C, 17A-D, 18A-C, 19A-C, 20A-D, 21A-E, 22A-C, and 23A-E show various user interfaces of the mobile application (or a web application). FIG. 16A shows a user interface 1600 displaying an icon 1602 corresponding to the mobile application. FIG. 16B shows a user interface 1604 during an initialization procedure, with the splash screen displayed. FIG. 16C shows a user interface 1606 after initialization procedure completes. The user interface 1606 includes a menu with multiple icons related to appointment, nurse location, directory, video chat, wound analysis, chart and website.

FIG. 17A shows a user interface 1700 corresponding to set pickup location feature. FIG. 17B shows a user interface 1702 containing a registration form. FIG. 17C shows a user interface 1704 with a form that allows a user to enter credit card data. FIG. 17D shows a user interface 1706 depicting a pickup location and a drop-off location.

FIG. 18A shows a user interface 1800 depicting the menu of nurses and physicians. FIG. 18B shows a user interface 1802 depicting an example of nurses, showing credentials, photo and distance. FIG. 18C. shows a user interface 1804 depicting an example of provider physician, showing credentials, photo and distance.

FIG. 19A shows a user interface 1900 depicting a portal logon screen to electronic medical record. FIG. 19B shows a user interface 1902 depicting a portrait view of a medical record. FIG. 19C shows a user interface 1904 depicting a landscape view of the mobile access to the medical record. Further, the user interface 1904 may depict the provider physician, showing credentials, photo and distance.

FIG. 20A shows a user interface 2000 depicting a selection screen. FIG. 20B shows a user interface 2002 depicting types of services. FIG. 20C shows a user interface 2004 depicting details about an order wound consultation. FIG. 20D shows a user interface 2006 depicting an order for a webinar on wound care.

FIG. 21A shows a user interface 2100 depicting a registration form for a waiting room. FIG. 21B shows a user interface 2102 depicting a logon screen. FIG. 21C shows a user interface 2104 depicting a patient waiting for a provider. FIG. 21D shows a user interface 2106 depicting the acceptance of the provider. FIG. 21E shows a user interface 2108 depicting a chat message feature.

FIG. 22A shows a user interface 2200 for a web application depicting a waiting room patient entry. FIG. 22B shows a user interface 2202 for a web application depicting a provider screen remote computer. FIG. 22C shows a user interface 2204 for a web application depicting a waiting room patient listing.

FIG. 23A shows a user interface 2300 for a mobile application depicting a login screen. FIG. 23B shows a user interface 2302 depicting a photo of a wound on a particular day. FIG. 23C shows a user interface 2304 depicting a login screen for accessing web tissue analysis reports. FIG. 23D shows a user interface 2306 depicting a gallery of wound photos. FIG. 23E shows a user interface 2308 depicting analysis of a wound.

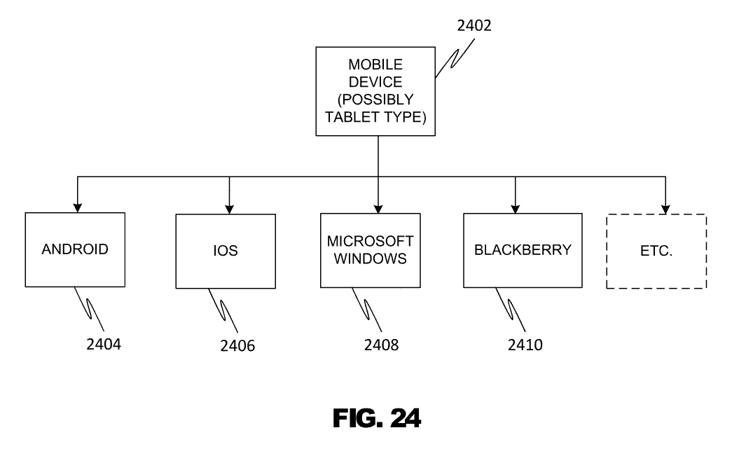

FIG. 24 is a block diagram showing operating systems supported, in accordance with some embodiments. As shown, the various users may use mobile device 2402. The mobile device 2402 may support one or more operating systems including, but not limited to, Android™ 2404, iOS™ 2406, Microsoft Windows™ 2408 and Blackberry™ 2410.

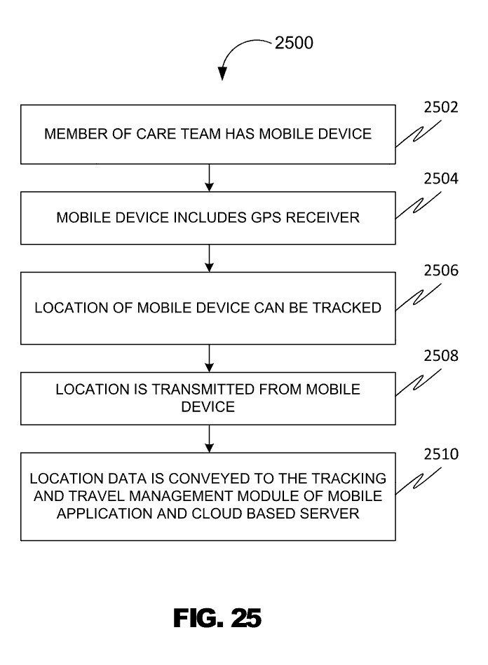

FIG. 25 is a flowchart of a mobile device tracking method 2500, in accordance with some embodiments. At 2502, the method 2500 incudes a member of care team accessing a mobile device. Then, at 2504, the method 2500 includes the mobile device initiating a GPS receiver. Thereafter, at 2506, the method 2500 includes tracking the location of the mobile device. Next, at 2508, the method 2500 includes transmitting the location from the mobile device. However, at 2510, the method 2500 includes conveying the location data to a tracking and travel management module of mobile application and cloud based server.

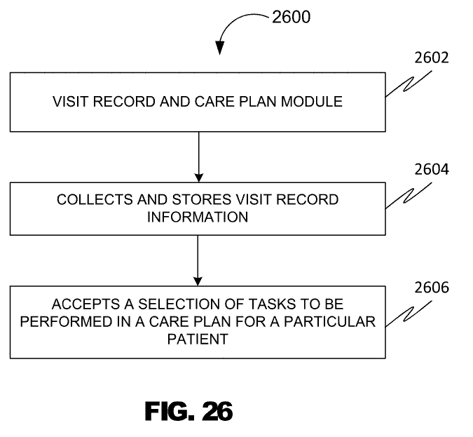

FIG. 26 is a flowchart related to a visit recording method 2600, in accordance with some embodiments. At 2602, the method 2600 includes initiating a visit record and care plan module. Then, at 2604, the method 2600 includes collecting and storing the visit record information. Further, at 2606, the method 2600 includes accepting a selected of tasks to be performed in a care plan for a particular patient.

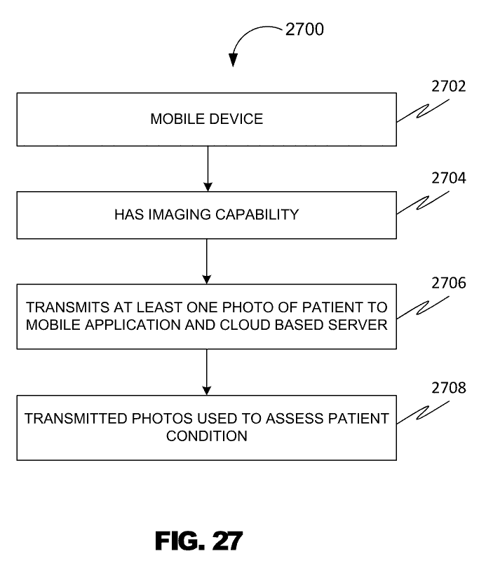

FIG. 27 is a flowchart related to an image transmission method 2700, in accordance with some embodiments. At 2702, the method 2700 includes initiating a mobile device. Then, at 2704, the method 2700 includes initiating a camera of the mobile device and clicking at least one photo of a wound. Thereafter, at 2706, the method 2700 includes transmitting the at least one photo via the mobile application to the cloud based server. Thereafter, at 2708, the method 2700 includes assessing patient condition based on the transmitted photos.

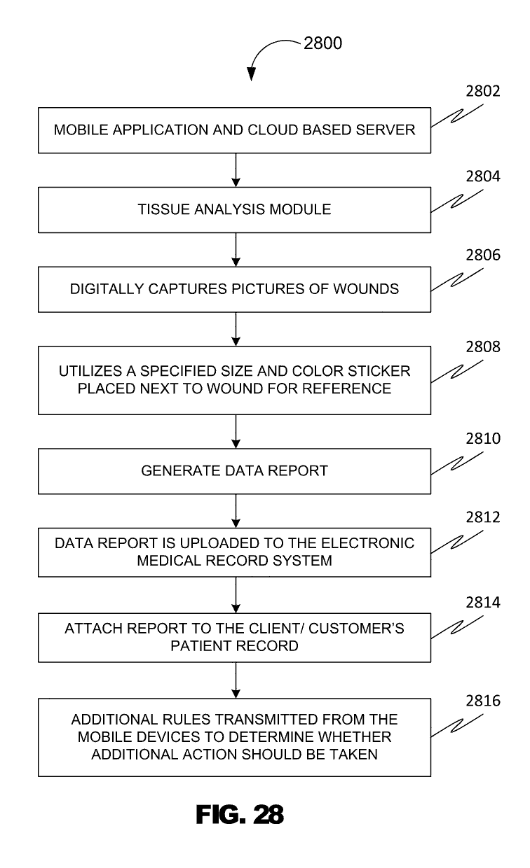

FIG. 28 is a flowchart related to an image capture method 2800, in accordance with some embodiments. At 2802, the method 2800 includes initiating the mobile application and establishing connection with the cloud based server. Further, at 2804, the method 2800 includes initiating a tissue analysis module. Yet further, at 2806, the method 2800 includes digitally capturing one or more photos of wounds. Moreover, at 2808, the method 2800 includes utilizing a specified size and color sticker placed next to wound for reference. Then, at 2810, the method 2800 includes generating a data report. Next, at 2812, the method 2800 includes uploading the data report to the electronic medical record system. Thereafter, at 2814, the method 2800 includes attaching the data report to the client/customers patient record. Further, at 2816, transmitting additional rules from the mobile devices to determine whether additional action should be taken.

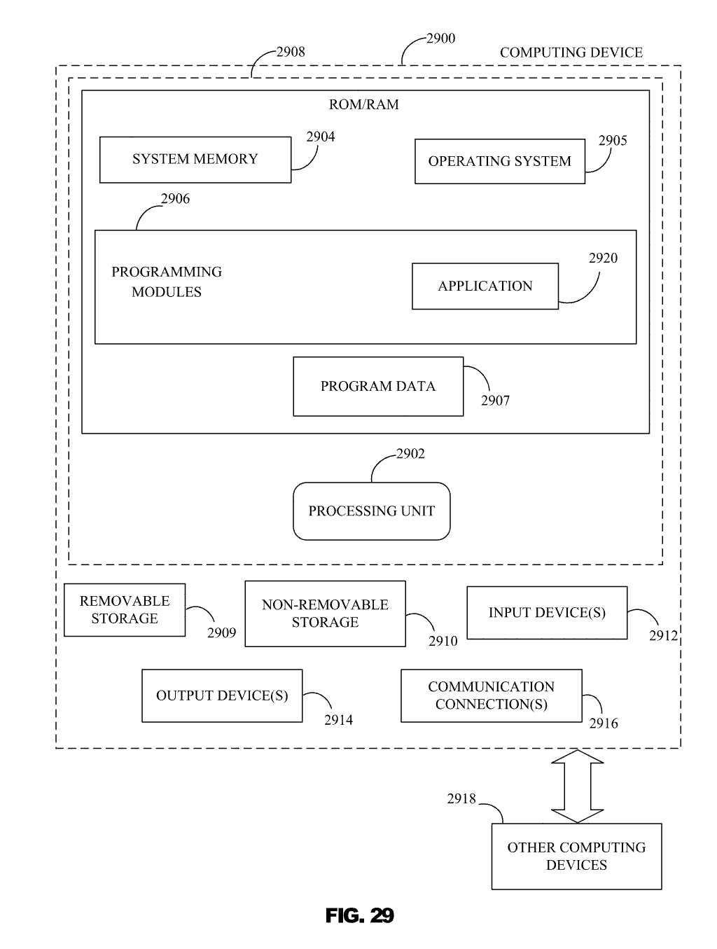

With reference to FIG. 29, a system consistent with an embodiment of the disclosure may include a computing device or cloud service, such as computing device 2900. In a basic configuration, computing device 2900 may include at least one processing unit 2902 and a system memory 2904. Depending on the configuration and type of computing device, system memory 2904 may comprise, but is not limited to, volatile (e.g. random access memory (RAM)), non-volatile (e.g. read-only memory (ROM)), flash memory, or any combination. System memory 2904 may include operating system 2905, one or more programming modules 2906, and may include a program data 2907. Operating system 2905, for example, may be suitable for controlling computing device 2900's operation. In one embodiment, programming modules 2906 may include image encoding module, machine learning module and image classifying module. Furthermore, embodiments of the disclosure may be practiced in conjunction with a graphics library, other operating systems, or any other application program and is not limited to any particular application or system. This basic configuration is illustrated in FIG. 29 by those components within a dashed line 2908.

Computing device 2900 may have additional features or functionality. For example, computing device 2900 may also include additional data storage devices (removable and/or non-removable) such as, for example, magnetic disks, optical disks, or tape. Such additional storage is illustrated in FIG. 29 by a removable storage 2909 and a non-removable storage 2910. Computer storage media may include volatile and nonvolatile, removable and non-removable media implemented in any method or technology for storage of information, such as computer-readable instructions, data structures, program modules, or other data. System memory 2904, removable storage 2909, and non-removable storage 2910 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2900. Any such computer storage media may be part of device 2900. Computing device 2900 may also have input device(s) 2912 such as a keyboard, a mouse, a pen, a sound input device, a touch input device, etc. Output device(s) 2914 such as a display, speakers, a printer, etc. may also be included. The aforementioned devices are examples and others may be used.

Computing device 2900 may also contain a communication connection 2916 that may allow device 2900 to communicate with other computing devices 2918, such as over a network in a distributed computing environment, for example, an intranet or the Internet. Communication connection 2916 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2904, including operating system 2905. While executing on processing unit 2902, programming modules 2906 (e.g., application 2920) may perform processes including, for example, one or more stages of methods 300, 400, 500, 600, 700, 800, 900, 1200, 1300, 1400, 1500, 2500, 2600, 2700 and 2800 as described above. The aforementioned process is an example, and processing unit 2902 may perform other processes. Other programming modules that may be used in accordance with embodiments of the present disclosure may include image encoding applications, machine learning application, image classifiers etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention.

I/We claim:

1. A method of facilitating wound care management of a subject, the method comprising:
receiving, using a communication device, a request for wound management from a user device;
receiving, using the communication device, at least one image of a wound of the subject from the user device;
analyzing, using a processing device, the at least one image of the wound;
determining, using the processing device, at least one medical condition of the wound based on the analyzing;
generating, using the processing device, at least one work schedule for at least one healthcare-provider based on at least one of the request and the at least one medical condition; and
transmitting, using the communication device, the at least one work schedule to at least one healthcare-provider device associated with the at least one healthcare-provider.

2. The method of claim 1 further comprising:
transmitting, using the communication device, a service request to a plurality of healthcare-provider devices associated with a plurality of health care providers;
receiving, using the communication device, at least one response from at least one of the plurality of healthcare-provider devices; and
identifying, using the processing device, the at least one healthcare-provider based on the at least one response.

3. The method of claim 1 further comprising:
receiving, using the communication device, tracking data from the at least one healthcare-provider device associated with the at least one healthcare-provider;
comparing, using the processing device, at least one schedule parameter associated with the at least one work schedule with a corresponding tracking data; and
validating, using the processing device, the at least one work schedule based on a result of the comparing.

4. The method of claim 3 further comprising:
transmitting, using the communication device, an electronic form to the at least one healthcare-provider device, wherein the electronic form is based on at least one of the request and the medical condition; and
receiving, using the communication device, form data from the at least one healthcare-provider device, wherein the form data is entered into the electronic form by the at least one healthcare-provider;
analyzing, using the processing device, the form data based on at least one rule, wherein validating the at least one work schedule is further based on the analyzing of the form data.

5. The method of claim 1 further comprising:
receiving, using the communication device, at least one current location of the at least one healthcare-provider device;
generating, using the processing device, at least one route associated with the at least one work schedule based on the at least one current location; and
transmitting, using the communication device, the at least one route to the at least one healthcare-provider device.

6. The method of claim 1 further comprising establishing, using the communication device, an encrypted communication session between a healthcare-provider device and the user device based on at least one of the request and the medical condition.

7. The method of claim 1 further comprising:
determining, using the processing device, at least one wound characteristic associated with the wound based on analysis of the at least one image of the wound, wherein the at least one image comprises representation of a reference object, and wherein the analysis is based on at least one physical characteristic of the reference object;
identifying, using the processing device, at least one dressing product based on the at least one wound characteristic; and
transmitting, using the communication device, indication of the at least one dressing product to at least one of the user device and the at least one healthcare-provider device.

8. The method of claim 7 further comprising:
identifying, using the processing device, treatment instructions based on at least one of the at least one wound characteristic and the medical condition;
transmitting, using the communication device, the treatment instructions to at least one of the user device and the at least one healthcare-provider device.

9. The method of claim 7 further comprising initiating, using the processing device, dispatching of the at least one dressing product to a location associated with the subject.

10. The method of claim 9 further comprising determining, using the processing device, a time schedule associated with the dispatching, wherein the time schedule comprises indication of a plurality of times and a plurality of dressing products to be dispatched at the plurality of times.

11. A system for facilitating wound care management of a subject, the system comprising:
a communication device configured for
receiving a request for wound management from a user device;
receiving at least one image of a wound of the subject from the user device;
transmitting at least one work schedule to at least one healthcare-provider device associated with at least one healthcare-provider; and
a processing device configured for:
analyzing the at least one image of the wound;
determining at least one medical condition of the wound based on the analyzing;
generating the at least one work schedule for the at least one healthcare-provider based on at least one of the request and the at least one medical condition.

12. The system of claim 11, wherein the communication device is further configured for:
transmitting a service request to a plurality of healthcare-provider devices associated with a plurality of health care providers; and
receiving at least one response from at least one of the plurality of healthcare-provider devices, wherein the processing device is further configured for identifying the at least one healthcare-provider based on the at least one response.

13. The system of claim 11, wherein the communication device is further configured for receiving tracking data from the at least one healthcare-provider device associated with the at least one healthcare-provider, wherein the processing device is further configured for:
comparing at least one schedule parameter associated with the at least one work schedule with a corresponding tracking data; and
validating the at least one work schedule based on a result of the comparing.

14. The system of claim 13, wherein the communication device is further configured for:
transmitting an electronic form to the at least one healthcare-provider device, wherein the electronic form is based on at least one of the request and the medical condition; and
receiving form data from the at least one healthcare-provider device, wherein the form data is entered into the electronic form by the at least one healthcare-provider, wherein the processing device is further configured for analyzing the form data based on at least one rule, wherein validating the at least one work schedule is further based on the analyzing of the form data.

15. The system of claim 11, wherein the communication device is further configured for:
receiving at least one current location of the at least one healthcare-provider device; and
transmitting at least one route to the at least one healthcare-provider device, wherein the processing device is further configured for generating at least one route associated with the at least one work schedule based on the at least one current location.

16. The system of claim 11, wherein the communication device is further configured for establishing an encrypted communication session between a healthcare-provider device and the user device based on at least one of the request and the medical condition.

17. The system of claim 11, wherein the processing device is further configured for:
determining at least one wound characteristic associated with the wound based on analysis of the at least one image of the wound, wherein the at least one image comprises representation of a reference object, wherein the analysis is based on at least one physical characteristic of the reference object;
identifying at least one dressing product based on the at least one wound characteristic; and
transmitting indication of the at least one dressing product to at least one of the user device and the at least one healthcare-provider device.

18. The system of claim 17, wherein the processing device is further configured for identifying treatment instructions based on at least one of the at least one wound characteristic and the medical condition, wherein the communication device is further configured for transmitting the treatment instructions to at least one of the user device and the at least one healthcare-provider device.

19. The system of claim 17, wherein the processing device is further configured for initiating dispatching of the at least one dressing product to a location associated with the subject.

20. The system of claim 19, wherein the processing device is further configured for determining a time schedule associated with the dispatching, wherein the time schedule comprises indication of a plurality of times and a plurality of dressing products to be dispatched at the plurality of times.
